(19) United States
(12) Patent Application Publication
(10) Pub. No.: US 2023/0157596 A1
(43) Pub. Date: May 25, 2023

(54) ANALYTE SENSORS FOR SENSING GLUTAMATE AND METHODS OF USING THE SAME

(71) Applicant: Abbott Diabetes Care Inc., Alameda, CA (US)

(72) Inventors: Rifat Emrah OZEL, San Jose, CA (US); Stephen OJA, Oakland, CA (US); Benjamin J. FELDMAN, Berkeley, CA (US)

(73) Assignee: Abbott Diabetes Care Inc., Alameda, CA (US)

(21) Appl. No.: 18/055,057

(22) Filed: Nov. 14, 2022

Related U.S. Application Data
(60) Provisional application No. 63/281,243, filed on Nov. 19, 2021.

(51) Int. Cl. A61B 5/1486; A61B 5/145

(52) U.S. Cl. CPC A61B 5/14865; A61B 5/1451; A61B 5/14546

(57) ABSTRACT

The present disclosure provides an analyte sensor for use in detecting glutamate. In certain embodiments, a glutamate-responsive active site of a presently disclosed analyte sensor includes a glutamate oxidase and a redox mediator disposed upon a surface of a working electrode. The present disclosure further provides methods for detecting glutamate using the disclosed analyte sensors.

### FIELD

The subject matter described herein relates to analyte sensors for sensing glutamate and methods of using the same.

### BACKGROUND

The detection of various analytes within an individual can sometimes be vital for monitoring the condition of their health as deviations from normal analyte levels can be indicative of a physiological condition. For example, monitoring glucose levels can enable a person suffering from diabetes to take appropriate corrective action to avoid significant physiological harm from hypoglycemia, hyperglycemia or ketoacidosis. Other analytes, such as glutamate, can be desirable to monitor for other physiological conditions.

Glutamate or glutamic acid is one of the excitatory signaling molecules in the central nervous system (CNS) and contributes to neurotransmission in the brain. Under physiological conditions, the blood/plasma levels of glutamate remain relatively stable. After a traumatic brain injury (TBI) such as stroke and intracerebral hemorrhage, however, plasma glutamate levels can be significantly increased and remain elevated for an extended period resulting in a worse neurological outcome and other complications such as acute lung injury (ALI). Therefore, it is critical to monitor glutamate levels after a traumatic brain injury to achieve a better prognosis and neurological outcome.

Analyte monitoring in an individual can take place periodically or continuously over a period of time. Periodic analyte monitoring can take place by withdrawing a sample of bodily fluid, such as blood or urine, at set time intervals and analyzing ex vivo. Periodic, ex vivo analyte monitoring can be sufficient to determine the physiological condition of many individuals. However, ex vivo analyte monitoring can be inconvenient or painful in some instances. Moreover, there is no way to recover lost data if an analyte measurement is not obtained at an appropriate time. Continuous analyte monitoring can be conducted using one or more sensors that remain at least partially implanted within a tissue of an individual, such as dermally, subcutaneously or intravenously, so that analyses can be conducted in vivo. Implanted sensors can collect analyte data on-demand, at a set schedule, or continuously, depending on an individual's particular health needs and/or previously measured analyte levels. Analyte monitoring with an in vivo implanted sensor can be a more desirable approach for individuals having severe analyte dysregulation and/or rapidly fluctuating analyte levels, although it can also be beneficial for other individuals as well.

Enzyme-based amperometric sensors configured for assaying glucose continuously in vivo have been developed and refined over recent years to aid in monitoring the health of diabetic individuals. However, analyte sensors configured for detecting analytes other than glucose in vivo are known but are considerably less refined at present. Accordingly, there is a need in the art for sensors for detecting analytes like glutamate in vivo.

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

To achieve these and other advantages and in accordance with the purpose of the disclosed subject matter, as embodied and broadly described, the disclosed subject matter provides an analyte sensor for detecting glutamate. In certain embodiments, an analyte sensor of the present disclosure includes a sensor tail including at least a first working electrode, a glutamate-responsive active area disposed upon a surface of the first working electrode and a mass transport limiting membrane permeable to glutamate that overcoats at least a portion of the glutamate-responsive active area. In certain embodiments, the glutamate-responsive active area includes a glutamate oxidase and an electron transfer agent.

The present disclosure further provides methods for detecting glutamate. In certain embodiments, the method can include providing an analyte sensor that includes (a) a sensor tail including at least a first working electrode, (b) a glutamate-responsive active area disposed upon a surface of the first working electrode, wherein the glutamate-responsive active area includes a glutamate oxidase and an electron transfer agent; and (c) a mass transport limiting membrane permeable to glutamate that overcoats at least the glutamate-responsive active area. In certain embodiments, the method further includes applying a potential to the first working electrode, obtaining a first signal at or above an oxidation-reduction potential of the glutamate-responsive active area, the first signal being proportional to a concentration of glutamate in a fluid contacting the glutamate-responsive active area, and correlating the first signal to the concentration of glutamate in the fluid. In certain embodiments, the fluid is interstitial fluid.

In certain embodiments, the glutamate-responsive area further includes a polymer. For example, but not by way of limitation, the electron transfer agent and/or glutamate oxidase are crosslinked to the polymer in the glutamate-responsive active area. In certain embodiments, the electron transfer agent and/or glutamate oxidase are covalently bonded to the polymer in the glutamate-responsive active area. In certain embodiments, the glutamate-responsive active area further includes a stabilizer, e.g., an albumin.

In certain embodiments, the mass transport limiting membrane includes a polyurethane or a copolymer thereof. In certain embodiments, the mass transport limiting membrane further comprises an ion-exchange polymer, e.g., a perfluorosulfonic acid polymer, or a polyvinylpyridine-based polymer.

In certain embodiments, an analyte sensor of the present disclosure further includes a second working electrode and a second active area disposed upon a surface of the second working electrode and responsive to a second analyte differing from glutamate. In certain embodiments, the second active area includes at least one enzyme responsive to the second analyte. In certain embodiments, a second portion of the mass transport limiting membrane overcoats the second active area or a second mass transport limiting membrane overcoats the second active area.

In certain embodiments, the analyte is implanted in a subject at risk of having or has a neurological condition. In certain embodiments, the neurological condition is a brain injury. In certain embodiments, the brain injury is a traumatic brain injury. In certain embodiments, the traumatic brain injury is a stroke and/or an intracerebral hemorrhage.

In certain embodiments, the analyte sensor is implanted in a subject for about 15 days. In certain embodiments, the analyte sensor retains at least about 90% sensitivity during its use.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, without departing from the scope of this disclosure.

FIG. 1 shows a diagram of an illustrative sensing system that can incorporate an analyte sensor of the present disclosure.

FIGS. 2A-2C show cross-sectional diagrams of analyte sensors including a single active area.

FIGS. 3A-3C show cross-sectional diagrams of analyte sensors including two active areas.

FIG. 4 shows a cross-sectional diagram of an analyte sensor including two active areas.

FIGS. 5A-5C show perspective views of analyte sensors including two active areas upon separate working electrodes.

FIG. 6 shows a diagram of a particular enzyme system that can be used for detecting glutamate according to the present disclosure.

FIG. 7 shows a sensor current (nA) versus time (hour) plot of an exemplary glutamate sensor of the present disclosure.

FIG. 8 shows an illustrative plot of sensor current response (nA) versus glutamate concentration (μM) for the glutamate sensor of FIG. 7 at Day 1 and Day 5.

FIG. 9 shows an illustrative plot of sensor current response (nA) versus time (hour) for a glutamate sensor comprising alternative membrane compositions.

FIG. 10 shows an illustrative plot of sensor current (nA) versus glutamate concentration (μM) for a glutamate sensor comprising alternative membrane compositions.

FIG. 11 shows the stability of a glutamate sensor at Day 1, Day 6 and Day 12 as measured by sensor current (nA) versus glutamate concentration (mM).

FIG. 12 shows an illustrative plot of sensor current (nA) versus glutamate concentration (mM) for glutamate sensors coated with a HydroMed™ D1 membrane or a HydroMed™ D7 membrane.

FIG. 13 shows an illustrative plot of sensor current (nA) versus time (hour) for glutamate sensors coated with membranes composed of HydroMed™ D1 and polyvinylpyridine or HydroMed™ D1 and 10Q5.

FIG. 14 shows an illustrative plot of sensor current (nA) versus glutamate concentration (04) for glutamate sensors coated with membranes composed of HydroMed™ D1 and polyvinylpyridine or HydroMed™ D1 and 10Q5.

### DETAILED DESCRIPTION

The present disclosure is directed to analyte sensors employing one or more enzymes for the detection of glutamate. In certain embodiments, the present disclosure further provides analyte sensors employing multiple enzymes for detecting two different analytes, e.g., employing multiple enzymes for the detection of glutamate and a second analyte. Depending on sensor configuration, the analyte sensors of the present disclosure can be configured to detect one analyte, e.g., glutamate, or multiple analytes simultaneously or near simultaneously. The present disclosure further provides methods of detecting one or more analytes, e.g., glutamate, using the disclosed analyte sensors.

The present disclosure provides sensor chemistries and mass transport limiting membranes suitable for detecting glutamate with good response stability and sensitivity over a range of glutamate concentrations. In certain embodiments, the glutamate sensors of the present disclosure are stable for up to about 11 days, about 12 days, about 13 days, about 14 days, about 15 days, about 16 days, about 17 days, about 18 days, about 19 days or about 20 days after initial use, e.g., after implantation. In certain embodiments, a glutamate sensor of the present disclosure exhibits a loss of sensitivity of less than about 20%, less than about 19%, less than about 18%, less than about 17%, less than about 16%, less than about 15%, less than about 14%, less than about 13%, less than about 14%, less than about 13%, less than about 12%, less than about 11%, less than about 10%, less than about 9%, less than about 8%, less than about 7%, less than about 6% or less than about 5% during the use of the sensor. In certain embodiments, a continuous glutamate sensor of the present disclosure exhibits a loss in sensitivity of less than about 10% during the use of the sensor, e.g., over a wearing period of about 12 days. In certain embodiments, a continuous glutamate sensor of the present disclosure exhibits a loss of less than about 15%, e.g., 13%, in sensitivity during the use of the sensor, e.g., over a wearing period of about 15 days.

For clarity, but not by way of limitation, the detailed description of the presently disclosed subject matter is divided into the following subsections:

I. Definitions; and

II. Analyte Sensors;
1. General Structure of Analyte Sensors;
2. Enzymes;
3. Redox Mediators;
4. Polymeric Backbone;
5. Mass Transport Limiting Membranes; and
6. Manufacturing;

III. Methods of Use.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, e.g., within 5-fold or within 2-fold, of a value.

The term “biological fluid,” as used herein, refers to any bodily fluid or bodily fluid derivative in which the analyte can be measured. Non-limiting examples of a biological fluid include dermal fluid, interstitial fluid, plasma, blood, lymph, synovial fluid, cerebrospinal fluid, saliva, bronchoalveolar lavage, amniotic fluid, sweat, tears or the like. In certain embodiments, the biological fluid is dermal fluid or interstitial fluid.

As used herein, the term “redox mediator” refers to an electron transfer agent for carrying electrons between an analyte or an analyte-reduced or analyte oxidized enzyme and an electrode, either directly, or via one or more additional electron transfer agents. In certain embodiments, redox mediators that include a polymeric backbone can also be referred to as “redox polymers.”

The term “electrolysis,” as used herein, refers to electrooxidation or electroreduction of a compound either directly at an electrode or via one or more electron transfer agents (e.g., redox mediators or enzymes).

The term “reference electrode” as used herein, can refer to either reference electrodes or electrodes that function as both, a reference and a counter electrode. Similarly, the term “counter electrode,” as used herein, can refer to both, a counter electrode and a counter electrode that also functions as a reference electrode.

As used herein, the term “homogenous membrane” refers to a membrane comprising a single type of membrane polymer.

As used herein, the term “multi-component membrane” refers to a membrane comprising two or more types of membrane polymers.

As used herein, the term “single-component membrane” refers to a membrane comprising one type of membrane polymer.

As used herein, the term “ion-exchange polymer” refers to a polymer that can exchange ions (cations or anions) with ionic components in solution.

As used herein, the term “polyvinylpyridine-based polymer” refers to a polymer or copolymer that comprises polyvinylpyridine (e.g., poly(2-vinylpyridine) or poly(4-vinylpyridine)) or a derivative thereof.

### II. Analyte Sensors

1. General Structure of Analyte Sensors

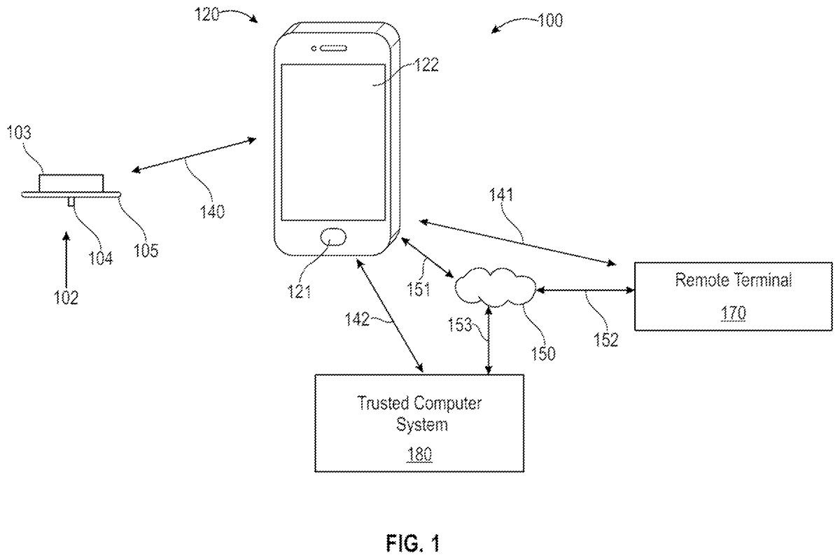

Before describing the analyte sensors of the present disclosure and their components in further detail, a brief overview of suitable in vivo analyte sensor configurations and sensor systems employing the analyte sensors will be provided so that the embodiments of the present disclosure can be better understood. FIG. 1 shows a diagram of an illustrative sensing system that can incorporate an analyte sensor of the present disclosure. As shown, sensing system 100 includes sensor control device 102 and reader device 120 that are configured to communicate with one another over a local communication path or link 140, which can be wired or wireless, uni- or bi-directional, and encrypted or non-encrypted. Reader device 120 can constitute an output medium for viewing analyte concentrations and alerts or notifications determined by sensor 104 or a processor associated therewith, as well as allowing for one or more user inputs, according to certain embodiments. Reader device 120 can be a multi-purpose smartphone or a dedicated electronic reader instrument. While only one reader device 120 is shown, multiple reader devices 120 can be present in certain instances. Reader device 120 can also be in communication with remote terminal 170 and/or trusted computer system 180 via communication path(s)/link(s) 141 and/or 142, respectively, which also can be wired or wireless, uni- or bi-directional, and encrypted or non-encrypted. Reader device 120 can also or alternately be in communication with network 150 (e.g., a mobile telephone network, the internet, or a cloud server) via communication path/link 151. Network 150 can be further communicatively coupled to remote terminal 170 via communication path/link 152 and/or trusted computer system 180 via communication path/link 153. Alternately, sensor 104 can communicate directly with remote terminal 170 and/or trusted computer system 180 without an intervening reader device 120 being present. For example, but not by the way of limitation, sensor 104 can communicate with remote terminal 170 and/or trusted computer system 180 through a direct communication link to network 150, according to certain embodiments, as described in U.S. Patent Application Publication 2011/0213225 and incorporated herein by reference in its entirety. Any suitable electronic communication protocol can be used for each of the communication paths or links, such as near field communication (NFC), radio frequency identification (RFID), BLUETOOTH® or BLUETOOTH® Low Energy protocols, WiFi, or the like. Remote terminal 170 and/or trusted computer system 180 can be accessible, according to certain embodiments, by individuals other than a primary user who have an interest in the user's analyte levels. Reader device 120 can include display 122 and optional input component 121. Display 122 can include a touch-screen interface, according to certain embodiments.

Sensor control device 102 includes sensor housing 103, which can house circuitry and a power source for operating sensor 104. Optionally, the power source and/or active circuitry can be omitted. A processor (not shown) can be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to certain embodiments.

Sensor 104 is adapted to be at least partially inserted into a tissue of interest, such as within the dermal or subcutaneous layer of the skin. Sensor 104 can include a sensor tail of sufficient length for insertion to a desired depth in a given tissue. The sensor tail can include at least one working electrode. In certain configurations, the sensor tail can include an active area for detecting an analyte. A counter electrode can be present in combination with the at least one working electrode. Particular electrode configurations upon the sensor tail are described in more detail below.

The active area can be configured for detecting a particular analyte. For example, but not by way of limitation, the disclosed analyte sensors include at least one active area configured to detect glutamate. In certain embodiments, a sensor of the present disclosure includes two active areas, where each active area is configured to detect a different analyte. Alternatively, the two active areas can be configured to detect the same analyte. In certain embodiments, a first active area can be configured to detect glutamate and a second active area can be configured to detect glutamate or a second analyte different from glutamate.

In certain embodiments of the present disclosure, one or more analytes can be monitored in any biological fluid of interest such as dermal fluid, interstitial fluid, plasma, blood, lymph, synovial fluid, cerebrospinal fluid, saliva, bronchoalveolar lavage, amniotic fluid, or the like. In certain particular embodiments, analyte sensors of the present disclosure can be adapted for assaying dermal fluid or interstitial fluid to determine a concentration of one or more analytes in vivo. In certain embodiments, the biological fluid is interstitial fluid.

Referring still to FIG. 1, sensor 104 can automatically forward data to reader device 120. For example but not by the way of limitation, analyte concentration data (i.e., glutamate concentration) can be communicated automatically and periodically, such as at a certain frequency as data is obtained or after a certain time period has passed, with the data being stored in a memory until transmittal (e.g., every minute, five minutes, or other predetermined time period). In certain other embodiments, sensor 104 can communicate with reader device 120 in a non-automatic manner and not according to a set schedule. For example, but not by the way of limitation, data can be communicated from sensor 104 using RFID technology when the sensor electronics are brought into communication range of reader device 120. Until communicated to reader device 120, data can remain stored in a memory of sensor 104. Thus, a user does not have to maintain close proximity to reader device 120 at all times, and can instead upload data at a convenient time. In certain other embodiments, a combination of automatic and non-automatic data transfer can be implemented. For example, and not by the way of limitation, data transfer can continue on an automatic basis until reader device 120 is no longer in communication range of sensor 104.

An introducer can be present transiently to promote introduction of sensor 104 into a tissue. In certain illustrative embodiments, the introducer can include a needle or similar sharp. As would be readily recognized by a person skilled in the art, other types of introducers, such as sheaths or blades, can be present in alternative embodiments. More specifically, the needle or other introducer can transiently reside in proximity to sensor 104 prior to tissue insertion and then be withdrawn afterward. While present, the needle or other introducer can facilitate insertion of sensor 104 into a tissue by opening an access pathway for sensor 104 to follow. For example, and not by the way of limitation, the needle can facilitate penetration of the epidermis as an access pathway to the dermis to allow implantation of sensor 104 to take place, according to one or more embodiments. After opening the access pathway, the needle or other introducer can be withdrawn so that it does not represent a sharps hazard. In certain embodiments, suitable needles can be solid or hollow, beveled or non-beveled, and/or circular or non-circular in cross-section. In more particular embodiments, suitable needles can be comparable in cross-sectional diameter and/or tip design to an acupuncture needle, which can have a cross-sectional diameter of about 250 microns. However, suitable needles can have a larger or smaller cross-sectional diameter if needed for certain particular applications.

In certain embodiments, a tip of the needle (while present) can be angled over the terminus of sensor 104, such that the needle penetrates a tissue first and opens an access pathway for sensor 104. In certain embodiments, sensor 104 can reside within a lumen or groove of the needle, with the needle similarly opening an access pathway for sensor 104. In either case, the needle is subsequently withdrawn after facilitating sensor insertion.

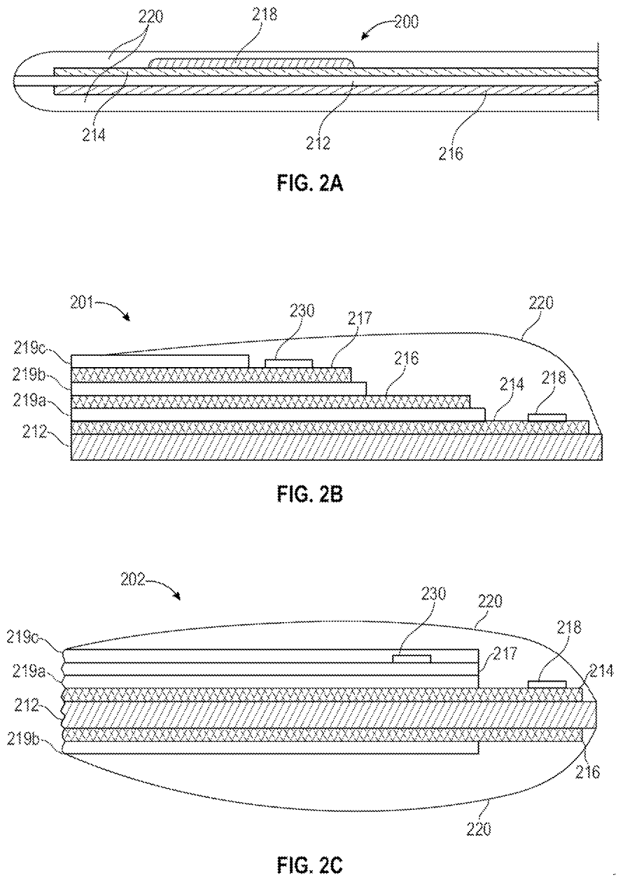

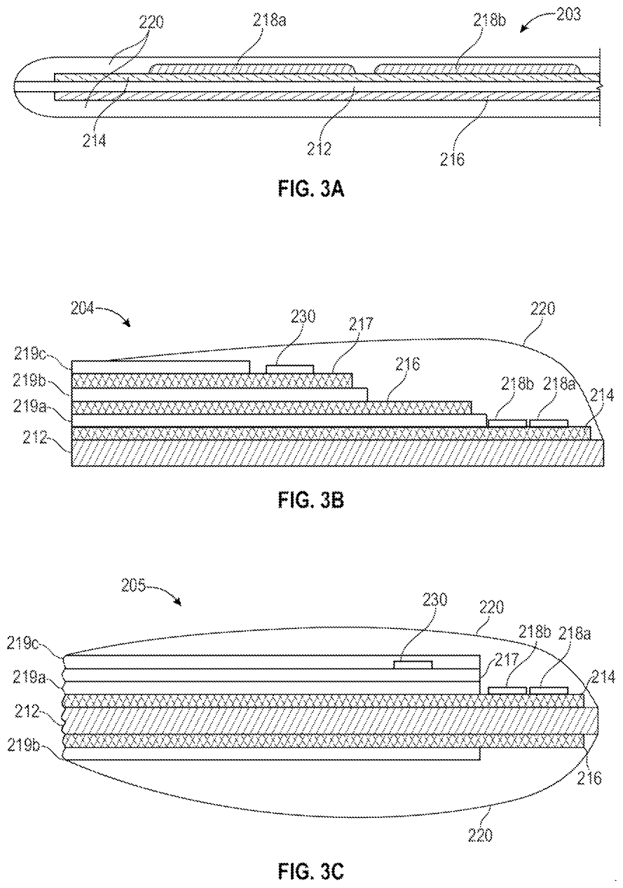

Sensor configurations featuring a single active area that is configured for the detection of a corresponding single analyte can employ two-electrode or three-electrode detection motifs, as described further herein in reference to FIGS. 2A-2C. Sensor configurations featuring two different active areas for detection of separate analytes, either upon separate working electrodes or upon the same working electrode, are described separately thereafter in reference to FIGS. 3A-5C. Sensor configurations having multiple working electrodes can be particularly advantageous for incorporating two different active areas within the same sensor tail, since the signal contribution from each active area can be determined more readily.

When a single working electrode is present in an analyte sensor, three-electrode sensor configurations can include a working electrode, a counter electrode, and a reference electrode. Related two-electrode sensor configurations can include a working electrode and a second electrode, in which the second electrode can function as both a counter electrode and a reference electrode (i.e., a counter/reference electrode). The various electrodes can be at least partially stacked (layered) upon one another and/or laterally spaced apart from one another upon the sensor tail. Suitable sensor configurations can be substantially flat in shape, substantially cylindrical in shape or any other suitable shape. In any of the sensor configurations disclosed herein, the various electrodes can be electrically isolated from one another by a dielectric material or similar insulator.

Analyte sensors featuring multiple working electrodes can similarly include at least one additional electrode. When one additional electrode is present, the one additional electrode can function as a counter/reference electrode for each of the multiple working electrodes. When two additional electrodes are present, one of the additional electrodes can function as a counter electrode for each of the multiple working electrodes and the other of the additional electrodes can function as a reference electrode for each of the multiple working electrodes.

FIG. 2A shows a diagram of an illustrative two-electrode analyte sensor configuration, which is compatible for use in the disclosure herein. As shown, analyte sensor 200 includes substrate 212 disposed between working electrode 214 and counter/reference electrode 216. Alternately, working electrode 214 and counter/reference electrode 216 can be located upon the same side of substrate 212 with a dielectric material interposed in between (configuration not shown). Active area 218 is disposed as at least one layer upon at least a portion of working electrode 214. Active area 218 can include multiple spots or a single spot configured for detection of an analyte, as discussed further herein.

Referring still to FIG. 2A, membrane 220 overcoats at least active area 218. In certain embodiments, membrane 220 can also overcoat some or all of working electrode 214 and/or counter/reference electrode 216, or the entirety of analyte sensor 200. One or both faces of analyte sensor 200 can be overcoated with membrane 220. Membrane 220 can include one or more polymeric membrane materials having capabilities of limiting analyte flux to active area 218 (i.e., membrane 220 is a mass transport limiting membrane having some permeability for the analyte of interest). According to the disclosure herein, and further described below, membrane 220 can be crosslinked with a branched crosslinker in certain particular sensor configurations. For example, but not by way of limitation, membrane 220 is crosslinked with a crosslinking agent as described herein. The composition and thickness of membrane 220 can vary to promote a desired analyte flux to active area 218, thereby providing a desired signal intensity and stability. Analyte sensor 200 can be operable for assaying an analyte by any of coulometric, amperometric, voltammetric, or potentiometric electrochemical detection techniques.

FIGS. 2B and 2C show diagrams of illustrative three-electrode analyte sensor configurations, which are also compatible for use in the disclosure herein. Three-electrode analyte sensor configurations can be similar to that shown for analyte sensor 200 in FIG. 2A, except for the inclusion of additional electrode 217 in analyte sensors 201 and 202 (FIGS. 2B and 2C). With additional electrode 217, counter/reference electrode 216 can then function as either a counter electrode or a reference electrode, and additional electrode 217 fulfills the other electrode function not otherwise accounted for. Working electrode 214 continues to fulfill its original function. Additional electrode 217 can be disposed upon either working electrode 214 or electrode 216, with a separating layer of dielectric material in between. For example, and not by the way of limitation, as depicted in FIG. 2B, dielectric layers 219a, 219b and 219c separate electrodes 214, 216 and 217 from one another and provide electrical isolation. Alternatively, at least one of electrodes 214, 216 and 217 can be located upon opposite faces of substrate 212, as shown in FIG. 2C. Thus, in certain embodiments, electrode 214 (working electrode) and electrode 216 (counter electrode) can be located upon opposite faces of substrate 212, with electrode 217 (reference electrode) being located upon one of electrodes 214 or 216 and spaced apart therefrom with a dielectric material. Reference material layer 230 (e.g., Ag/AgCl) can be present upon electrode 217, with the location of reference material layer 230 not being limited to that depicted in FIGS. 2B and 2C. As with sensor 200 shown in FIG. 2A, active area 218 in analyte sensors 201 and 202 can include multiple spots or a single spot. Additionally, analyte sensors 201 and 202 can be operable for assaying an analyte by any of coulometric, amperometric, voltammetric, or potentiometric electrochemical detection techniques.

Like analyte sensor 200, membrane 220 can also overcoat active area 218, as well as other sensor components, in analyte sensors 201 and 202, thereby serving as a mass transport limiting membrane. In certain embodiments, the additional electrode 217 can be overcoated with membrane 220. Although FIGS. 2B and 2C have depicted electrodes 214, 216 and 217 as being overcoated with membrane 220, it is to be recognized that in certain embodiments only working electrode 214 is overcoated. Moreover, the thickness of membrane 220 at each of electrodes 214, 216 and 217 can be the same or different. As in two-electrode analyte sensor configurations (FIG. 2A), one or both faces of analyte sensors 201 and 202 can be overcoated with membrane 220 in the sensor configurations of FIGS. 2B and 2C, or the entirety of analyte sensors 201 and 202 can be overcoated. Accordingly, the three-electrode sensor configurations shown in FIGS. 2B and 2C should be understood as being non-limiting of the embodiments disclosed herein, with alternative electrode and/or layer configurations remaining within the scope of the present disclosure.

FIG. 3A shows an illustrative configuration for sensor 203 having a single working electrode with two different active areas disposed thereon. FIG. 3A is similar to FIG. 2A, except for the presence of two active areas upon working electrode 214: first active area 218a and second active area 218b, which are responsive to different analytes and are laterally spaced apart from one another upon the surface of working electrode 214. Active areas 218a and 218b can include multiple spots or a single spot configured for detection of each analyte. The composition of membrane 220 can vary or be compositionally the same at active areas 218a and 218b. First active area 218a and second active area 218b can be configured to detect their corresponding analytes at working electrode potentials that differ from one another, as discussed further below.

FIGS. 3B and 3C show cross-sectional diagrams of illustrative three-electrode sensor configurations for sensors 204 and 205, respectively, each featuring a single working electrode having first active area 218a and second active area 218b disposed thereon. FIGS. 3B and 3C are otherwise similar to FIGS. 2B and 2C and can be better understood by reference thereto. As with FIG. 3A, the composition of membrane 220 can vary or be compositionally the same at active areas 218a and 218b.

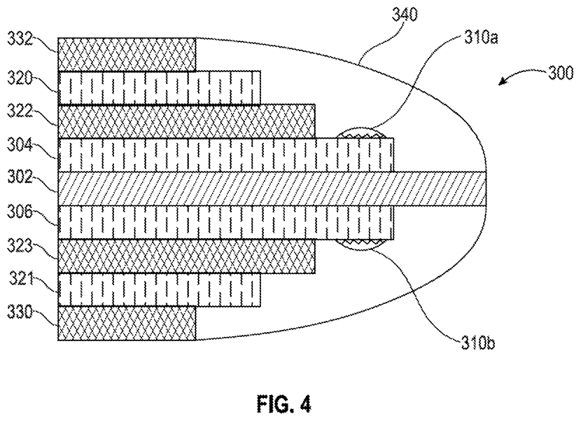

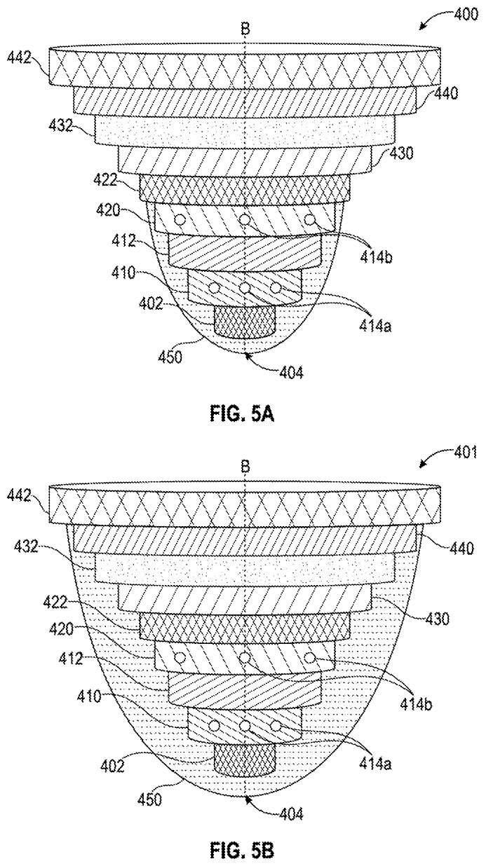

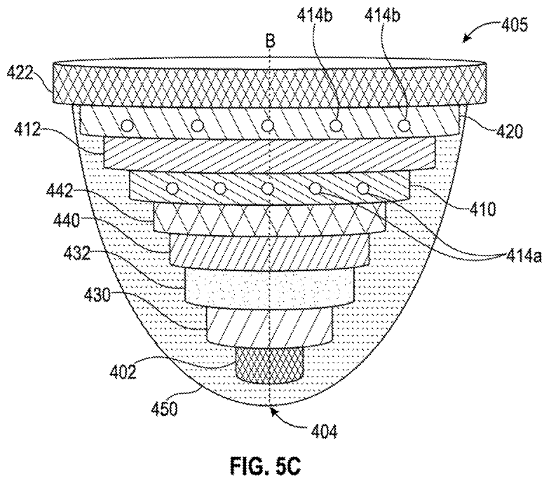

Illustrative sensor configurations having multiple working electrodes, specifically two working electrodes, are described in further detail in reference to FIGS. 4-5C. Although the following description is primarily directed to sensor configurations having two working electrodes, it is to be appreciated that more than two working electrodes can be incorporated through extension of the disclosure herein. Additional working electrodes can be used to impart additional sensing capabilities to the analyte sensors beyond just a first analyte and a second analyte, e.g., for the detection of a third and/or fourth analyte.

FIG. 4 shows a cross-sectional diagram of an illustrative analyte sensor configuration having two working electrodes, a reference electrode and a counter electrode, which is compatible for use in the disclosure herein. As shown, analyte sensor 300 includes working electrodes 304 and 306 disposed upon opposite faces of substrate 302. First active area 310a is disposed upon the surface of working electrode 304, and second active area 310b is disposed upon the surface of working electrode 306. Counter electrode 320 is electrically isolated from working electrode 304 by dielectric layer 322, and reference electrode 321 is electrically isolated from working electrode 306 by dielectric layer 323. Outer dielectric layers 330 and 332 are positioned upon reference electrode 321 and counter electrode 320, respectively. Membrane 340 can overcoat at least active areas 310a and 310b, according to various embodiments, with other components of analyte sensor 300 or the entirety of analyte sensor 300 optionally being overcoated with membrane 340. Membrane 340 can be continuous but vary compositionally upon active area 310a and/or upon active area 310b in order to afford different permeability values for differentially regulating the analyte flux at each location. For example, different membrane formulations can be sprayed and/or printed onto the opposing faces of analyte sensor 300. Dip coating techniques can also be appropriate, particularly for depositing at least a portion of a bilayer membrane upon one of active areas 310a and 310b. In certain embodiments, membrane 340 can be the same or vary compositionally at active areas 310a and 310b. For example, but not by way of limitation, membrane 340 can include a bilayer overcoating active area 310a and be a homogeneous membrane overcoating active area 310b, or membrane 340 can include a bilayer overcoating active area 310b and be a homogeneous membrane overcoating active area 310a. In certain embodiments, an analyte sensor can include more than one membrane 340, e.g., two or more membranes. For example, but not by way of limitation, an analyte sensor can include a membrane that overcoats the one or more active areas, e.g., 310a and 310b, and an additional membrane that overcoats the entire sensor as shown in FIG. 4.

Like analyte sensors 200, 201 and 202, analyte sensor 300 can be operable for assaying glutamate by any of coulometric, amperometric, voltammetric, or potentiometric electrochemical detection techniques.

Alternative sensor configurations having multiple working electrodes and differing from the configuration shown in FIG. 4 can feature a counter/reference electrode instead of separate counter and reference electrodes 320, 321, and/or feature layer and/or membrane arrangements varying from those expressly depicted. For example, and not by the way of limitation the positioning of counter electrode 320 and reference electrode 321 can be reversed from that depicted in FIG. 4. In addition, working electrodes 304 and 306 need not necessarily reside upon opposing faces of substrate 302 in the manner shown in FIG. 4.

Although suitable sensor configurations can feature electrodes that are substantially planar in character, it is to be appreciated that sensor configurations featuring non-planar electrodes can be advantageous and particularly suitable for use in the disclosure herein. In particular, substantially cylindrical electrodes that are disposed concentrically with respect to one another can facilitate deposition of a mass transport limiting membrane, as described hereinbelow. FIGS. 5A-5C show perspective views of analyte sensors featuring two working electrodes that are disposed concentrically with respect to one another. It is to be appreciated that sensor configurations having a concentric electrode disposition but lacking a second working electrode are also possible in the present disclosure.

FIG. 5A shows a perspective view of an illustrative sensor configuration in which multiple electrodes are substantially cylindrical and are disposed concentrically with respect to one another about a central substrate. As shown, analyte sensor 400 includes central substrate 402 about which all electrodes and dielectric layers are disposed concentrically with respect to one another. In particular, working electrode 410 is disposed upon the surface of central substrate 402, and dielectric layer 412 is disposed upon a portion of working electrode 410 distal to sensor tip 404. Working electrode 420 is disposed upon dielectric layer 412, and dielectric layer 422 is disposed upon a portion of working electrode 420 distal to sensor tip 404. Counter electrode 430 is disposed upon dielectric layer 422, and dielectric layer 432 is disposed upon a portion of counter electrode 430 distal to sensor tip 404. Reference electrode 440 is disposed upon dielectric layer 432, and dielectric layer 442 is disposed upon a portion of reference electrode 440 distal to sensor tip 404. As such, exposed surfaces of working electrode 410, working electrode 420, counter electrode 430, and reference electrode 440 are spaced apart from one another along longitudinal axis B of analyte sensor 400.

Referring still to FIG. 5A, first active areas 414a and second active areas 414b, which are responsive to different analytes or the same analyte, are disposed upon the exposed surfaces of working electrodes 410 and 420, respectively, thereby allowing contact with a fluid to take place for sensing. Although active areas 414a and 414b have been depicted as three discrete spots in FIG. 5A, it is to be appreciated that fewer or greater than three spots, including a continuous layer of active area, can be present in alternative sensor configurations.

In FIG. 5A, sensor 400 is partially coated with membrane 450 upon working electrodes 410 and 420 and active areas 414a and 414b disposed thereon. FIG. 5B shows an alternative sensor configuration in which the substantial entirety of sensor 401 is overcoated with membrane 450. Membrane 450 can be the same or vary compositionally at active areas 414a and 414b. For example, membrane 450 can include a bilayer overcoating active areas 414a and be a homogeneous membrane overcoating active areas 414b.

It is to be further appreciated that the positioning of the various electrodes in FIGS. 5A and 5B can differ from that expressly depicted. For example, the positions of counter electrode 430 and reference electrode 440 can be reversed from the depicted configurations in FIGS. 5A and 5B. Similarly, the positions of working electrodes 410 and 420 are not limited to those that are expressly depicted in FIGS. 5A and 5B. FIG. 5C shows an alternative sensor configuration to that shown in FIG. 5B, in which sensor 405 contains counter electrode 430 and reference electrode 440 that are located more proximal to sensor tip 404 and working electrodes 410 and 420 that are located more distal to sensor tip 404. Sensor configurations in which working electrodes 410 and 420 are located more distal to sensor tip 404 can be advantageous by providing a larger surface area for deposition of active areas 414a and 414b (five discrete sensing spots illustratively shown in FIG. 5C), thereby facilitating an increased signal strength in some cases. Similarly, central substrate 402 can be omitted in any concentric sensor configuration disclosed herein, wherein the innermost electrode can instead support subsequently deposited layers.

Several parts of the sensor are further described below.

2. Enzymes

An active area of a presently disclosed analyte sensor can be configured for detecting one or more analytes, e.g., glutamate. In certain embodiments, an analyte sensor of the present disclosure can include more than one active area, where each active area is configured to detect the same analyte or different analytes. The analyte sensors of the present disclosure include one or more active areas configured to detect glutamate. In certain embodiments, an analyte sensor of the present disclosure can further include one or more active areas configured to detect a second analyte other than glutamate.

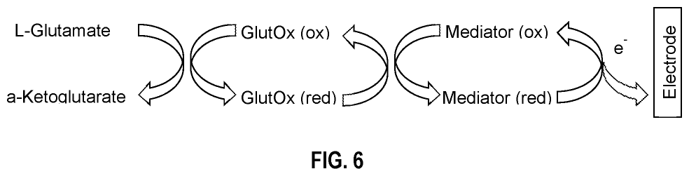

In certain embodiments, an analyte sensor of the present disclosure can include one or more glutamate-responsive areas. In certain embodiments, a glutamate-responsive area can include one or more enzymes for detecting glutamate. For example, but not by way of limitation, a glutamate-responsive area can include a glutamate oxidase as shown in FIG. 6. In certain embodiments, a glutamate-responsive active area contains a glutamate oxidase (“GlutOx” in FIG. 6) that converts L-glutamate into α-ketoglutarate (also referred to as “α-ketoglutaric acid”) and reduces glutamate oxidase. The reduced form of the glutamate oxidase can then transfer electron(s) to a redox mediator, which in turn can then be oxidized at an anode, i.e., the working electrode. The electrons transferred during this reaction provide the basis for glutamate detection at the working electrode. The electrochemical signal obtained can then be correlated to the amount of glutamate that was initially present in the sample.

In certain embodiments, an analyte sensor of the present disclosure can include a sensor tail comprising at least one working electrode and one or more glutamate-responsive active areas disposed upon the surface of the working electrode, where the glutamate-responsive active area includes a glutamate oxidase. In certain embodiments, the glutamate oxidase is immobilized in the glutamate-responsive active area, e.g., by covalent bonding to a polymer present within the glutamate-responsive active area.

In certain embodiments, the glutamate-responsive active area is disposed upon a portion of a working electrode. For example, but not by way of limitation, the glutamate-responsive active area is disposed upon a portion of the working electrode in a spotted pattern, e.g., two or more spots on the working electrode. In certain embodiments, the glutamate-responsive active area is disposed upon a portion of the working electrode in a slotted pattern. In certain embodiments, the glutamate-responsive active area is disposed upon the entire length of the working electrode or in a continuous pattern on the working electrode. In certain embodiments, a glutamate-responsive active area has an area of about 0.01 mm² to about 2.0 mm², e.g., about 0.1 mm² to about 1.0 mm² or about 0.2 mm² to about 0.5 mm².

In certain embodiments, the glutamate-responsive active area can further include a stabilizer, e.g., for stabilizing the enzyme. For example, but not by way of limitation, the stabilizer can be an albumin, e.g., a serum albumin. Non-limiting examples of serum albumins include bovine serum albumin and human serum albumin. In certain embodiments, the stabilizer is a human serum albumin. In certain embodiments, the stabilizer is a bovine serum albumin.

In certain embodiments, the glutamate-responsive active area can include a ratio of stabilizer to glutamate oxidase from about 40:1 to about 1:40, e.g., from about 35:1 to about 1:35, from about 30:1 to about 1:30, from about 25:1 to about 1:25, from about 20:1 to about 1:20, from about 15:1 to about 1:15, from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, from about 2:1 to about 1:2 or about 1:1. In certain embodiments, the glutamate-responsive active area can include a ratio of stabilizer to glutamate oxidase from about 1:1 to about 1:10, e.g., from about 1:1 to about 1:9, from about 1:1 to about 1:8, from about 1:1 to about 1:7, from about 1:1 to about 1:6, from about 1:1 to about 1:5, from about 1:2 to about 1:9, from about 1:3 to about 1:8, from about 1:3 to about 1:7 or from about 1:4 to about 1:6.

In certain embodiments, an analyte sensor can include two working electrodes, e.g., a first active area disposed on a first working electrode and a second active area disposed on a second working electrode. In certain embodiments, an analyte sensor disclosed herein can feature a glutamate-responsive active area and a second active area for detecting an analyte different from glutamate. For example, but not by way of limitation, such analyte sensors can include a sensor tail with at least a first working electrode and a second working electrode, a glutamate-responsive active area disposed upon a surface of the first working electrode and a second active area, e.g., a second enzyme-responsive active area, configured to detect a different analyte disposed upon a surface of the second working electrode. In certain embodiments, the additional analyte, e.g., second analyte, detected by an analyte sensor of the present disclosure can be glucose, lactate, ketone, creatinine and/or alcohol. In certain embodiments, when the sensor is configured to detect two or more analytes, detection of each analyte can include applying a potential to each working electrode separately, such that separate signals are obtained from each analyte. The signal obtained from each analyte can then be correlated to an analyte concentration through use of a calibration curve or function, or by employing a lookup table. In certain particular embodiments, correlation of the analyte signal to an analyte concentration can be conducted through use of a processor.

In certain embodiments, the second enzyme-responsive active area, e.g., present on a second working electrode, of an analyte sensor of the present disclosure can include one or more enzymes that can be used to detect glucose. For example, but not by way of limitation, an analyte sensor of the present disclosure can include an active area that comprises one or more enzymes for detecting glucose, e.g., disposed on a second working electrode. In certain embodiments, the analyte sensor can include an active site comprising a glucose oxidase and/or a glucose dehydrogenase for detecting glucose.

In certain embodiments, the second enzyme-responsive active area, e.g., present on a second working electrode, of an analyte sensor of the present disclosure can include one or more enzymes that can be used to detect ketones. For example, but not by way of limitation, an analyte sensor of the present disclosure can include an active area that comprises one or more enzymes, e.g., an enzyme system, for detecting ketones, e.g., disposed on a second working electrode. In certain embodiments, the analyte sensor can include an active site comprising P-hydroxybutyrate dehydrogenase and/or diaphorase for detecting ketones.

In certain embodiments, the second enzyme-responsive active area, e.g., present on a second working electrode, of an analyte sensor of the present disclosure can include one or more enzymes that can be used to detect lactate. For example, but not by way of limitation, an analyte sensor of the present disclosure can include an active area that comprises one or more enzymes, e.g., an enzyme system, for detecting lactate, e.g., disposed on a second working electrode. In certain embodiments, the analyte sensor can include an active site comprising a lactate dehydrogenase and/or a lactate oxidase.

In certain embodiments, the second enzyme-responsive active area, e.g., present on a second working electrode, of an analyte sensor of the present disclosure can include one or more enzymes that can be used to detect alcohol. For example, but not by way of limitation, an analyte sensor of the present disclosure can include an active area that comprises one or more enzymes, e.g., an enzyme system, for detecting alcohol, e.g., disposed on a second working electrode. In certain embodiments, the analyte sensor can include an active site comprising an alcohol dehydrogenase.

In certain embodiments, the second enzyme-responsive active area, e.g., present on a second working electrode, of an analyte sensor of the present disclosure can include one or more enzymes that can be used to detect creatinine. For example, but not by way of limitation, an analyte sensor of the present disclosure can include an active area that comprises one or more enzymes, e.g., an enzyme system, for detecting creatinine, e.g., disposed on a second working electrode. In certain embodiments, the analyte sensor can include an active site comprising an amidohydrolase, creatine amidinohydrolase and/or sarcosine oxidase.

In certain other analyte sensor configurations, the first active area and the second active area can be disposed upon a single working electrode. A first signal can be obtained from the first active area, e.g., at a low potential, and a second signal containing a signal contribution from both active areas can be obtained at a higher potential. Subtraction of the first signal from the second signal can then allow the signal contribution arising from the second analyte to be determined. The signal contribution from each analyte can then be correlated to an analyte concentration in a similar manner to that described for sensor configurations having multiple working electrodes. In certain embodiments, when the glutamate-responsive active area and the second active area, e.g., a second analyte-responsive active area, configured to detect a different analyte are arranged upon a single working electrode in this manner, one of the active areas can be configured such that it can be interrogated separately to facilitate detection of each analyte. For example, either the glutamate-responsive active area or the second active area responsive to the second analyte can produce a signal independently of the other active area.

It is also to be appreciated that the sensitivity (output current) of the analyte sensors toward each analyte can be varied by changing the coverage (area or size) of the active areas, the area ratio of the active areas with respect to one another, the identity, thickness and/or composition of a mass transport limiting membrane overcoating the active areas. Variation of these parameters can be conducted readily by one having ordinary skill in the art once granted the benefit of the disclosure herein.

3. Redox Mediators

In certain embodiments, an analyte sensor disclosed herein can include an electron transfer agent, e.g., a redox mediator. In certain embodiments, one or more active areas of an analyte sensor disclosed herein can include an electron transfer agent, e.g., a redox mediator.

In certain embodiments, a glutamate-responsive active area can include one or more electron transfer agents. For example, but not way of limitation, an analyte sensor of the present disclosure can include a sensor tail with at least a first working electrode and a glutamate-responsive active area disposed upon a surface of the first working electrode, where the glutamate-responsive active area comprises a glutamate oxidase and an electron transfer agent.

In certain embodiments, an analyte sensor of the present disclosure can include two or more active areas, where each active area includes an electron transfer agent. For example, but not way of limitation, an analyte sensor of the present disclosure can include a sensor tail with at least a first working electrode and a second working electrode, a glutamate-responsive active area comprising a glutamate oxidase and a first electron transfer agent disposed upon a surface of the first working electrode and a second analyte-responsive active area comprising at least one enzyme responsive to the second analyte and a second electron transfer agent disposed upon a surface of the second working electrode. Alternatively, an analyte sensor of the present disclosure can include two or more active areas, where only one active area includes an electron transfer agent. For example, but not way of limitation, an analyte sensor of the present disclosure can include a sensor tail with at least a first working electrode and a second working electrode, a glutamate-responsive active area comprising a glutamate oxidase and an electron transfer agent disposed upon a surface of the first working electrode and a second analyte-responsive active area comprising at least one enzyme responsive to the second analyte disposed upon a surface of the second working electrode, where the second analyte-responsive active area does not include an electron transfer agent.

Suitable electron transfer agents for use in the analyte sensors of the present disclosure can facilitate conveyance of electrons to the adjacent working electrode after an analyte undergoes an enzymatic oxidation-reduction reaction within the corresponding active area, thereby generating a current that is indicative of the presence of that particular analyte. The amount of current generated is proportional to the quantity of analyte that is present.

In certain embodiments, suitable electron transfer agents can include electroreducible and electrooxidizable ions, complexes or molecules (e.g., quinones) having oxidation-reduction potentials that are a few hundred millivolts above or below the oxidation-reduction potential of the standard calomel electrode (SCE). In certain embodiments, the electron transfer agent can include osmium complexes and other transition metal complexes, such as those described in U.S. Pat. Nos. 6,134,461 and 6,605,200, which are incorporated herein by reference in their entirety. Additional examples of suitable redox mediators include those described in U.S. Pat. Nos. 6,736,957, 7,501,053 and 7,754,093, the disclosures of each of which are also incorporated herein by reference in their entirety. Other examples of suitable electron transfer agents include metal compounds or complexes of ruthenium, osmium, iron (e.g., polyvinylferrocene or hexacyanoferrate), or cobalt, including metallocene compounds thereof, for example. Suitable ligands for the metal complexes can also include, for example, bidentate or higher denticity ligands such as, for example, bipyridine, biimidazole, phenanthroline, or pyridyl(imidazole). Other suitable bidentate ligands can include, for example, amino acids, oxalic acid, acetylacetone, diaminoalkanes, or o-diaminoarenes. Any combination of monodentate, bidentate, tridentate, tetradentate or higher denticity ligands can be present in a metal complex to achieve a full coordination sphere. In certain embodiments, the electron transfer agent is an osmium complex. In certain embodiments, the electron transfer agent is osmium complexed with bidentate ligands.

In certain embodiments, electron transfer agents disclosed herein can comprise suitable functionality to promote covalent bonding to a polymer (also referred to herein as a polymeric backbone) within the active areas as discussed further below. For example, but not by way of limitation, an electron transfer agent for use in the present disclosure can include a polymer-bound electron transfer agent, e.g., a redox polymer. Suitable non-limiting examples of polymer-bound electron transfer agents include those described in U.S. Pat. Nos. 8,444,834, 8,268,143 and 6,605,201, the disclosures of which are incorporated herein by reference in their entirety. In certain embodiments, the electron transfer agent is a bidentate osmium complex bound to a polymer described herein, e.g., a polymeric backbone described in Section 4 below. In certain embodiments, the polymer-bound electron transfer agent shown in FIG. 3 of U.S. Pat. No. 8,444,834 (referred to as “X7”) can be used in a sensor of the present disclosure.

In certain embodiments, the glutamate-responsive active area can include a ratio of glutamate oxidase to redox mediator from about 10:1 to about 1:10, e.g., from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, from about 2:1 to about 1:2, from about 1.5:1 to about 1:1.5 or about 1:1.

4. Polymeric Backbone

In certain embodiments, one or more active sites for promoting analyte detection can include a polymer to which an enzyme and/or redox mediator is covalently bound. Any suitable polymeric backbone can be present in the active area for facilitating detection of an analyte through covalent bonding of the enzyme and/or redox mediator thereto. Non-limiting examples of suitable polymers within the active area include polyvinylpyridines, e.g., poly(4-vinylpyridine), and polyvinylimidazoles, e.g., poly(N-vinylimidazole) and poly(l-vinylimidazole), or a copolymer thereof, for example, in which quaternized pyridine groups serve as a point of attachment for the redox mediator or enzyme thereto. In certain embodiments, the polymer is a poly(4-vinylpyridine) polymer or a derivative thereof. Non-limiting polymers for use in the present disclosure are disclosed in U.S. Pat. No. 8,444,834.

Illustrative copolymers that can be suitable for inclusion in the active areas include those containing monomer units such as styrene, acrylamide, methacrylamide, or acrylonitrile, for example. In certain embodiments, the polymer is a copolymer of vinylpyridine and styrene. Additional non-limiting examples of polymers that can be present in the active area include those described in U.S. Pat. No. 6,605,200, incorporated herein by reference in its entirety, such as poly(acrylic acid), styrene/maleic anhydride copolymer, methylvinylether/maleic anhydride copolymer (GANTREZ™ polymer), poly(vinylbenzylchloride), poly(allylamine), polylysine, poly(-vinylpyridine) quaternized with carboxypentyl groups, and poly(sodium 4-styrene sulfonate). In certain embodiments where the analyte sensor includes two active sites, the polymer within each active area can be the same or different.

In certain embodiments, an enzyme of a given active area can be immobilized. In certain embodiments, an enzyme of an active area is covalently bonded to the polymer. Alternatively or additionally, the enzyme of an active area can be non-covalently associated with the polymer, such that the non-covalently bonded enzyme is physically retained within the polymer. In certain embodiments, glutamate oxidase present can be covalently bonded to a polymer within the glutamate-responsive active area of the disclosed analyte sensors. For example, but not by way of limitation, glutamate oxidase can be covalently bonded to a polymer of a redox mediator, i.e., redox polymer, within the glutamate-responsive active area of the disclosed analyte sensors. In certain embodiments, glutamate oxidase can be non-covalently associated with the polymer.

In certain particular embodiments, covalent bonding of the one or more enzymes and/or redox mediators to the polymer in a given active area can take place via crosslinking introduced with a suitable crosslinking agent. Suitable crosslinking agents for reaction with free amino groups in the enzyme (e.g., with the free side chain amine in lysine) can include crosslinking agents such as, for example, polyethylene glycol diglycidyl ether (PEGDGE) or other polyepoxides, cyanuric chloride, glutaraldehyde, N-hydroxysuccinimide, imidoesters, epichlorohydrin or derivatized variants thereof. In certain embodiments, the crosslinking agent is PEGDGE, e.g., having a number average molecular weight (Mn) from about 200 to 1,000, e.g., about 400. In certain embodiments, the crosslinking agent is PEGDGE 400. In certain embodiments, the crosslinking agent can be glutaraldehyde. Suitable crosslinking agents for reaction with free carboxylic acid groups in the enzyme can include, for example, carbodiimides. In certain embodiments, the crosslinking of the enzyme to the polymer is generally intermolecular. In certain embodiments, the crosslinking of the enzyme to the polymer is generally intramolecular.

5. Mass Transport Limiting Membranes

In certain embodiments, the analyte sensors disclosed herein further include a membrane that overcoats at least one active area, e.g., a first active area and/or a second active area, of the analyte sensor. In certain embodiments, the membrane is permeable to the analyte to be detected in the active area, e.g., glutamate. In certain embodiments, the membrane overcoats each of the active areas of an analyte sensor. Alternatively, a first membrane overcoats one of the active areas and a second membrane overcoats the second active area. Alternatively, a first membrane overcoats one of the active areas and a second membrane subsequently overcoats both the first and second active areas.

In certain embodiments, a membrane overcoating an analyte-responsive active area can function as a mass transport limiting membrane and/or to improve biocompatibility. A mass transport limiting membrane can act as a diffusion-limiting barrier to reduce the rate of mass transport of the analyte. For example, but not by way of limitation, limiting access of an analyte, e.g., a glutamate, to the analyte-responsive active area with a mass transport limiting membrane can aid in avoiding sensor overload (saturation), thereby improving detection performance and accuracy.

In certain embodiments, the mass transport limiting membrane can be homogeneous and can be single-component (e.g., contain a single membrane polymer or a copolymer of two or more polymers). Alternatively, the mass transport limiting membrane can be multi-component (e.g., contain two or more different membrane polymers, e.g., as a composite). In certain embodiments, the multi-component membrane can be present as a multilayered membrane, e.g., a bilayer membrane or a trilayer membrane. In certain embodiments, the multi-component membrane can be present as a homogeneous admixture of two or more membrane polymers. In certain embodiments, a homogeneous admixture can be deposited by combining the two or more membrane polymers in a solution and then depositing the solution upon a working electrode, e.g., by dip coating. In certain embodiments, a multi-layered membrane can be deposited onto analyte-responsive active area by depositing a first layer, e.g., by dip coating, and depositing a second layer onto the first layer, e.g., by dip coating, to generate a bilayer membrane. In certain embodiments, a third layer can be deposited onto the second layer, e.g., by dip coating, to generate a trilayer membrane.

In certain embodiments, a mass transport limiting membrane of the present disclosure can have a neutral charge. In certain embodiments, mass transport limiting membranes that have a neutral charge allow glutamate to diffuse easily from the interstitial fluid to the glutamate-responsive active area present on the working electrode to generate a concentration dependent current signal.

In certain embodiments, a mass transport limiting membrane of the present disclosure can include a hydrophilic polymer. For example, but not by way of limitation, the mass transport limiting membrane can be a single-component membrane or a multi-component membrane comprising a hydrophilic polymer.

In certain embodiments, the polymer can be polyvinylpyridine-co-polystyrene sulfonate or polyvinylimidazole-co-poly(n-isopropylacrylamide) as described herein.

In certain embodiments, the polymer is a polyurethane. For example, but not by way of limitation, the mass transport limiting membrane can be a single-component membrane or a multi-component membrane comprising a polyurethane. In certain embodiments, a polymer for use in the present disclosure, e.g., a polyurethane, is capable of absorbing from about 30% to about 95% of its weight in water, e.g., from about 30% to about 70%. In certain embodiments, the polyurethane is capable of absorbing at least about 30% of its weight in water. In certain embodiments, the polyurethane is capable of absorbing at least about 40% of its weight in water. In certain embodiments, the polyurethane is capable of absorbing at least about 50% of its weight in water. In certain embodiments, the polyurethane is capable of absorbing at least about 60% of its weight in water. In certain embodiments, the polyurethane is capable of absorbing at least about 70% of its weight in water. In certain embodiments, a polyurethane for use in the present disclosure is low heat curable. For example, but not by way of limitation, a polymer for use in the present disclosure, e.g., a polyurethane, is curable at a temperature from about 20° C. to about 90° C., e.g., about 25° C. to about 85° C., about 30° C. to about 80° C., about 35° C. to about 75° C., about 40° C. to about 70° C., about 45° C. to about 65° C., about 20° C. to about 70° C., about 20° C. to about 60° C., about 20° C. to about 50° C., about 30° C. to about 90° C., about 40° C. to about 90° C., about 50° C. to about 90° C., about 60° C. to about 90° C. or about 70° C. to about 90° C. In certain embodiments, the polymer for use in the present disclosure, e.g., a polyurethane, has a molecular weight from about 50 to about 500 kDa.

In certain embodiments, the polyurethane can be a commercially available hydrophilic polyurethane. In certain embodiments, hydrophilic polyurethanes can include a polyurethane of the HydroMed™ Series from AdvanSource biomaterials (Wilmington, Mass.). For example, but not by way of limitation, the commercially available hydrophilic polyurethane can include HydroMed™ D1, HydroMed™ D2, HydroMed™ D3, HydroMed™ D4, HydroMed™ D6, HydroMed™ D640, HydroMed™ D7, HydroMed™ Hydroslip C or a combination thereof. In certain embodiments, the polyurethane can comprise HydroMed™ D7. In certain embodiments, the polyurethane can comprise HydroMed™ D1.

In certain embodiments, the membrane, e.g., a single-component membrane or a multi-component membrane, can include a copolymer of a polyurethane. In certain embodiments, the membrane, e.g., a single-component membrane, can include a copolymer of a polyurethane and one or more additional polymers, e.g., a second polymer. In certain embodiments, the second polymer is a hydrophilic polymer. In certain embodiments, polyurethane can be copolymerized with a hydrophilic polymer such as, but not limited to, a polyether, a polyester, a polyalkene, a polyamine and a polyalkylene oxide. In certain embodiments, polyurethane is copolymerized with polyethylene oxide, polybutylene oxide, polypropylene oxide or polytetramethylene oxide.

In certain embodiments, the mass transport limiting membrane can be multi-component membrane. For example, but not by way of limitation, the mass transport limiting membrane can be a composite of two or more polymers, e.g., three or more, four or more or five or more polymers. In certain embodiments, the mass transport limiting membrane can be a composite of at least two polymers. In certain embodiments, the mass transport limiting membrane is a composite of a polyurethane polymer or copolymer thereof and a second polymer. In certain embodiments, the mass transport limiting membrane can be a homogeneous admixture of a polyurethane polymer or copolymer thereof and a second polymer. In certain embodiments, the mass transport limiting membrane can have a multilayered configuration, where each layer comprises a different polymer. For example, but not by way of limitation, the mass transport limiting membrane can be composed of at least two layers, where each layer comprises a different polymer. In certain embodiments, the mass transport limiting membrane can be composed of three layers, where at least two of the three layers comprise a different polymer.

In certain embodiments, the second polymer is a fluoropolymer. In certain embodiments, the second polymer for use in a mass transport limiting membrane of the present disclosure is an ion-exchange polymer. In certain embodiments, the ion-exchange polymer is a short side chain ion-exchange polymer. In certain embodiments, the ion-exchange polymer is a long side chain ion-exchange polymer. In certain embodiments, the ion-exchange polymer is a perfluorosulfonic acid (PFSA) polymer or a perfluorocarboxylic acid (PFCA) polymer. In certain embodiments, the ion-exchange polymer is a PF SA polymer. In certain embodiments, the ion-exchange polymer has a softening point greater than about 75° C., greater than about 80° C., greater than about 85° C., greater than about 90° C. or greater than about 95° C.

In certain embodiments, the ion-exchange polymer can be a commercially available polymer. For example, but not by way of limitation, the ion-exchange polymer can be a polymer of the AQUIVION® product line from Solvay and/or a polymer of the NAFION® product line from Sigma Aldrich. In certain embodiments, the short side chain ion-exchange polymer is an AQUIVION® product. In certain embodiments, the long side chain ion-exchange polymer is an NAFION® product.

In certain embodiments, the second polymer is a polyvinylpyridine-based polymer. In certain embodiments, the polyvinylpyridine can be poly(2-vinylpyridine) or poly(4-vinylpyridine). In certain embodiments, the second polymer can be a copolymer of a polyvinylpyridine-based polymer or derivative thereof. For example, but not by way of limitation, the second polymer can be a copolymer of a polyvinylpyridine-based polymer or derivative thereof and styrene. In certain embodiments, the styrene can be derivatized, e.g., sulfonated. In certain embodiments, the second polymer can be a polyvinylpyridine-co-styrene copolymer that is derivatized. In certain embodiments, the second polymer is polyvinylpyridine-co-polystyrene sulfonate. In certain embodiments, the polyvinylpyridine-co-polystyrene sulfonate polymer comprises from about 15% to about 50% of polystyrene sulfonate per molar content. Non-limiting examples of polyvinylpyridine-based polymers are disclosed in U.S. Patent Publication No. 2003/0042137 (e.g., at Formula 2b), the contents of which are incorporated by reference herein in its entirety. In certain embodiments, the second polymer can be the 10Q5 polymer as described in U.S. Pat. No. 8,761,857, the contents of which are incorporated by reference herein in its entirety. In certain embodiments, the polyvinylpyridine-based polymer has a molecular weight from about 50 to about 300 kDa.

In certain embodiments, the second polymer is a polyvinylimidazole-based polymer. In certain embodiments, the second polymer can be a copolymer of a polyvinylimidazole-based polymer or derivative thereof. For example, but not by way of limitation, the second polymer can be a copolymer of a polyvinylimidazole-based polymer or derivative thereof and isopropylacrylamide. In certain embodiments, the polyvinylimidazole-based polymer comprises from about 30% to about 70% of polyvinylimidazole per molar content. In certain embodiments, the second polymer is polyvinylimidazole-co-poly(n-isopropylacrylamide). In certain embodiments, the polyvinylimidazole-based polymer has a molecular weight from about 50 to about 300 kDa.

In certain embodiments, the membrane comprises at least about 10% of a polyurethane or a copolymer thereof. For example, but not by way of limitation, the membrane comprises at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or about 100% of a polyurethane or a copolymer thereof. In certain embodiments, the membrane comprises from about 1% to about 100% of a polyurethane or a copolymer thereof, e.g., from about 1% to about 95%, from about 1% to about 90%, from about 1% to about 85%, from about 1% to about 80%, from about 1% to about 75%, from about 1% to about 70%, from about 1% to about 65%, from about 1% to about 60%, from about 1% to about 55%, from about 1% to about 50%, from about 1% to about 45%, from about 1% to about 40%, from about 1% to about 35%, from about 1% to about 30%, from about 1% to about 25%, from about 1% to about 20%, from about 1% to about 15%, from about 1% to about 10%, from about 5% to about 100%, from about 10% to about 100%, from about 15% to about 100%, from about 20% to about 100%, from about 25% to about 100%, from about 30% to about 100%, from about 35% to about 100%, from about 40% to about 100%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%, from about 20% to about 80%, from about 30% to about 70% or from about 40% to about 60%. In certain embodiments, the membrane comprises from about 1% to about 30% of a polyurethane or a copolymer thereof.

In certain embodiments, the membrane, e.g., a multi-component membrane, comprises at least about 10% of an additional polymer. For example, but not by way of limitation, the membrane comprises at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98% or at least about 99% of an additional polymer, e.g., a second polymer, e.g., an ion-exchange polymer. In certain embodiments, the membrane comprises from about 1% to about 95% of a polyurethane or a copolymer thereof, e.g., from about 1% to about 90%, from about 1% to about 85%, from about 1% to about 80%, from about 1% to about 75%, from about 1% to about 70%, from about 1% to about 65%, from about 1% to about 60%, from about 1% to about 55%, from about 1% to about 50%, from about 1% to about 45%, from about 1% to about 40%, from about 1% to about 35%, from about 1% to about 30%, from about 1% to about 25%, from about 1% to about 20%, from about 1% to about 15%, from about 1% to about 10%, from about 5% to about 95%, from about 10% to about 95%, from about 15% to about 95%, from about 20% to about 95%, from about 25% to about 95%, from about 30% to about 95%, from about 35% to about 95%, from about 40% to about 95%, from about 45% to about 95%, from about 50% to about 95%, from about 55% to about 95%, from about 60% to about 95%, from about 65% to about 95%, from about 70% to about 95%, from about 75% to about 95%, from about 80% to about 95%, from about 85% to about 95%, from about 90% to about 95%, from about 95% to about 95%, from about 20% to about 80%, from about 30% to about 70% or from about 40% to about 60%. In certain embodiments, the membrane comprises from about 1% to about 30% of an additional polymer, e.g., a second polymer. For example, but not by way of limitation, the membrane comprises from about 1% to about 30%, e.g., from about 1% to about 25% or from about 1% to about 20%, of an ion-exchange polymer. In certain embodiments, the membrane comprises from about 1% to about 20% of an additional polymer, e.g., a second polymer. In certain embodiments, the membrane comprises from about 1% to about 10% of an additional polymer, e.g., a second polymer. In certain embodiments, the membrane comprises from about 1% to about 10%, e.g., from about 2% to about 9%, from about 3% to about 8%, from about 4% to about 8% or from about 5% to about 8%, of a polyvinylpyridine-based polymer or a copolymer thereof or a derivative of a polyvinylpyridine-based copolymer. In certain embodiments, the second polymer is present in a separate layer from the layer that comprises polyurethane.

In certain other embodiments, a membrane polymer overcoating one or more active areas can be crosslinked with a branched crosslinker, which can decrease the amount of extractables obtainable from the mass transport limiting membrane. Non-limiting examples of a branched crosslinker include branched glycidyl ether crosslinkers, e.g., including branched glycidyl ether crosslinkers that include three or more crosslinkable groups, such as polyethyleneglycol tetraglycidyl ether.

In certain embodiments, polydimethylsiloxane (PDMS) can be incorporated in any of the mass transport limiting membranes disclosed herein. For example, but not by way of limitation, PDMS can be incorporated into a multi-component mass transport limiting membrane disclosed herein. In certain embodiments, PDMS can be incorporated into a multi-component mass transport limiting membrane that comprises a polyvinylpyridine-based polymer or a copolymer thereof or a derivative of a polyvinylpyridine copolymer, e.g., polyvinylpyridine, 10Q5 and/or polyvinylpyridine-co-polystyrene sulfonate.

In certain embodiments, the composition of the mass transport limiting membrane disposed on an analyte sensor that has two active areas can be the same or different where the mass transport limiting membrane overcoats each active area. For example, but not by way of limitation, the portion of the mass transport limiting membrane overcoating the glutamate-responsive active area can be multi-component and/or the portion of the mass transport limiting membrane overcoating the second analyte-responsive active area can be single-component. Alternatively, the portion of the mass transport limiting membrane overcoating the glutamate-responsive active area can be single-component and/or the portion of the mass transport limiting membrane overcoating the second analyte-responsive active area can be multi-component.

In certain embodiments of the present disclosure, the glutamate-responsive active area can be overcoated with a single-component membrane comprising a polyurethane (or a copolymer thereof) and the second active area responsive to the second analyte can be overcoated with a multi-component membrane comprising a polyvinylpyridine and/or a polyvinylpyridine-co-styrene copolymer. Alternatively, the glutamate-responsive active area can be overcoated with a multi-component membrane comprising a polyurethane (or a copolymer thereof) and a second polymer, e.g., an ion-exchange polymer or a polyvinylpyridine-based polymer, either as a bilayer or trilayer membrane or a homogeneous admixture, and the active area responsive to the second analyte can be overcoated with a single-component membrane comprising a polyvinylpyridine or a polyvinylpyridine-co-styrene copolymer.

In certain embodiments, the portion of the mass transport limiting membrane overcoating the glutamate-responsive active area and the portion of the mass transport limiting membrane overcoating the second analyte-responsive active area can both be single-component but comprise different polymers. For example, but not by way of limitation, the glutamate-responsive active area can be overcoated with a single-component membrane comprising a polyurethane (or a copolymer thereof) and the second active area responsive to the second analyte can be overcoated with a single-component membrane comprising a polyvinylpyridine or a polyvinylpyridine-co-styrene copolymer.

In certain embodiments, the portion of the mass transport limiting membrane overcoating the glutamate-responsive active area and the portion of the mass transport limiting membrane overcoating the second analyte-responsive active area can both be multi-component but comprise different polymers. For example, but not by way of limitation, the glutamate-responsive active area can be overcoated with a multi-component membrane comprising a polyurethane (or a copolymer thereof) and a second polymer, e.g., an ion-exchange polymer or a polyvinylpyridine-based polymer, and the second active area responsive to the second analyte can be overcoated with a multi-component membrane comprising a polyvinylpyridine and/or a polyvinylpyridine-co-styrene copolymer.

In certain embodiments when a first active area and a second active area configured for assaying different analytes are disposed on separate working electrodes, the mass transport limiting membrane can have differing permeability values for the first analyte and the second analyte. For example, but not by way of limitation, the mass transport limiting membrane overcoating at least one of the active areas can include an admixture of a first membrane polymer and a second membrane polymer, a bilayer of the first membrane polymer (e.g., the first membrane) and the second membrane polymer (e.g., the second membrane) or a trilayer of the first membrane polymer (e.g., the first membrane), the second membrane polymer (e.g., the second membrane) and a third membrane polymer (e.g., a third membrane). In certain embodiments, the third membrane polymer is the same as the first or second membrane polymer. A homogeneous membrane can overcoat the active area not overcoated with the admixture or the bilayer, wherein the homogeneous membrane includes only one of the first membrane polymer or the second membrane polymer. Advantageously, the architectures of the analyte sensors disclosed herein readily allow a continuous membrane having a homogenous membrane portion to be disposed upon a first active area and a multi-component membrane portion to be disposed upon a second active area of the analyte sensors, thereby levelizing the permeability values for each analyte concurrently to afford improved sensitivity and detection accuracy. Continuous membrane deposition can take place through sequential dip coating operations in particular embodiments.

In certain embodiments, an analyte sensor described herein can comprise a sensor tail comprising at least a first working electrode, a first active area disposed upon a surface of the first working electrode and a mass transport limiting membrane permeable to the first analyte that overcoats at least the first active area. In certain embodiments, the first active area comprises an enzyme system responsive to a first analyte, e.g., glutamate, that comprises at least one enzyme responsive to the first analyte, e.g., glutamate oxidase. For example, but not by way of limitation, an analyte sensor described herein can comprise a sensor tail comprising at least a first working electrode, a glutamate-responsive active area comprising an enzyme system comprising glutamate oxidase disposed upon a surface of the first working electrode and a mass transport limiting membrane permeable to glutamate that overcoats the glutamate-responsive active area. In certain embodiments, the mass transport limiting membrane comprises polyurethane or a copolymer thereof. In certain embodiments, the mass transport limiting membrane further comprises a second polymer, e.g., an ion-exchange polymer or a polyvinylpyridine-based polymer.

In certain embodiments, an analyte sensor of the present disclosure can include a second active area configured for detecting the same analyte as the first active area or a different analyte. In certain embodiments, at least a portion of the mass transport limiting membrane that overcoats the first active area can overcoat the second active area. Alternatively or additionally, a second mass transport limiting membrane can be used to overcoat the second active area. In certain embodiments, at least a portion of the second mass transport limiting membrane that overcoats the second active area can overcoat the first active area. In certain embodiments, the mass transport limiting membrane that overcoats the first active area is of a different composition than the second mass transport limiting membrane.

In certain embodiments, the mass transport limiting membranes have a thickness, e.g., a total thickness, e.g., a dry thickness, of about 5 μm to about 100 μm, e.g., from about 10 μm to about 90 μm, from about 10 μm to about 80 μm, from about 10 μm to about 70 μm, from about 10 μm to about 60 μm, from about 10 μm to about 50 μm, from about 10 μm to about 40 μm, from about 10 μm to about 30 μm, from about 10 μm to about 20 μm, from about 10 μm to about 15 μm, from about 10 μm to about 100 μm, from about 20 μm to about 100 μm, from about 30 μm to about 100 μm, from about 40 μm to about 100 μm, from about 50 μm to about 100 μm, from about 60 μm to about 100 μm, from about 70 μm to about 100 μm, from about 80 μm to about 100 μm, from about 90 μm to about 100 μm, from about 20 μm to about 60 μm or from about 30 μm to about 50 μm.

In certain embodiments where the mass transport limiting membranes comprise a single polymer, e.g., a polyurethane, e.g., a single layer of one polymer, the mass transport limiting membranes can have a thickness, e.g., a total thickness, e.g., a dry thickness, from about 10 μm to about 30 μm. In certain embodiments where the mass transport limiting membranes comprise two or more polymers, e.g., a polyurethane and a second polymer (e.g., a single layer comprising the two polymers or a multilayered membrane comprising the two polymers), the mass transport limiting membranes can have a thickness, e.g., a total thickness, e.g., a dry thickness, from about 30 μm to about 50 μm. In certain embodiments, a polyurethane layer of a mass transport limiting membrane disclosed herein has a thickness, e.g., a dry thickness, from about 10 μm to about 30 μm. In certain embodiments, a layer comprising a second polymer as described herein, e.g., an ion-exchange polymer and/or a polyvinylpyridine-based polymer, can have a thickness, e.g., a dry thickness, from about 10 μm to about 40 μm, e.g., from about 15 μm to about 35 μm.

In certain embodiments, a mass transport limiting membrane of the present disclosure can comprise a single layer of a polyurethane polymer. In certain embodiments, a mass transport limiting membrane of the present disclosure can comprise two or more layers of a polyurethane polymer, e.g., two, three or four layers of a polyurethane polymer. In certain embodiments, a mass transport limiting membrane of the present disclosure can comprise a single layer of a second polymer as described herein, e.g., an ion-exchange polymer. In certain embodiments, a mass transport limiting membrane of the present disclosure can comprise two or more layers of a second polymer, e.g., two, three or four layers of a second polymer, e.g., an ion-exchange polymer. In certain embodiments, the mass transport limiting membrane can have alternating layers of a polyurethane polymer and layers of a second polymer.

6. Manufacturing

The present disclosure further provides methods for manufacturing the presently disclosed analyte sensors that includes one or more active sites. In certain embodiments, the method includes generating a working electrode, e.g., a carbon electrode, e.g., by carbon printing.

In certain embodiments, the method can further include adding a composition comprising one or more enzymes onto a surface of the working electrode to generate an active site on the working electrode, e.g., in a pattern as described herein. For example, but not by way of limitation, the composition can include a glutamate oxidase. In certain embodiments, the composition can further include a crosslinking agent, e.g., polyethylene glycol diglycidyl ether, a buffer, e.g., HEPES (N-2-hydroxyethylpiperazine-N-2-ethane sufonic acid), and/or a stabilizer, e.g., a serum albumin. In certain embodiments, the composition can further include a redox mediator, e.g., a redox polymer. In certain embodiments, the method can further include curing the enzyme composition, e.g., thermal curing.

In certain embodiments, the method can further include adding a one or more membrane compositions on top of the cured enzyme composition, e.g., by dip coating. In certain embodiments, the membrane composition can include a single polymer, e.g., a polyurethane or a copolymer thereof. In certain embodiments, the membrane composition can include two or more polymers. For example, but not by way of limitation, the membrane composition can include a first polymer, e.g., a polyurethane or a copolymer thereof, and a second polymer, e.g., an ion-exchange polymer, e.g., as admixture or a multilayered membrane.

In certain embodiments, the method can include adding a first layer comprising a polyurethane or a copolymer thereof on top of the cured enzyme composition, e.g., by dip coating. In certain embodiments, the method can include adding a layer comprising a second polymer on top of the first layer comprising a polyurethane or a copolymer thereof, e.g., by dip coating. In certain embodiments, the method can include adding a second layer comprising a polyurethane or a copolymer thereof on top of the second polymer layer, e.g., by dip coating, to generate a trilayered membrane composition.

In certain embodiments, the method can include curing the membrane composition.

### III. Methods of Use

The present disclosure further provides methods of using the analyte sensors disclosed herein. In certain embodiments, the present disclosure provides methods for detecting glutamate in a subject in need thereof. In certain embodiments, the subject in need of glutamate monitoring can be a subject that is at risk of developing or has developed one or more disorders and/or conditions associated with glutamate dysregulation. For example, but not by way of limitation, the subject in need of glutamate monitoring can be a subject that is at risk of developing or has developed one or more disorders and/or conditions associated with elevated levels of glutamate as described herein. In certain embodiments, the subject in need of glutamate monitoring can be a subject that is at risk of developing or has developed one or more disorders and/or conditions associated with a glutamate deficiency as described herein.

In certain embodiments, a glutamate sensor of the present disclosure can be used to continuously monitor glutamate levels in a subject at risk of having or has a neurological disorder or injury, e.g., Parkinson's disease, multiple sclerosis (MS), Alzheimer's disease, stroke, amyotrophic lateral sclerosis or Lou Gehrig's disease (ALS) and traumatic brain injuries. Additional examples of diseases and disorders associated with glutamate dysregulation are disclosed in Li et al., Frontiers in Psychiatry 9:767 (2019); Guerriero et al., Curr. Neurol. Neurosci. Rep. 15:27 (2015) and Mliladinovic et al., Biomolecules 5(4):3112-3141 (2015), the contents of which are hereby incorporated by reference in their entireties.

In certain embodiments, a method for detecting glutamate, e.g., in a subject in need thereof, includes: (i) providing an analyte sensor including: (a) a sensor tail including at least a first working electrode; (b) a glutamate-responsive active area disposed upon a surface of the first working electrode and responsive to glutamate, where the glutamate-responsive active area comprises a glutamate oxidase and, optionally, a first polymer and/or an electron transfer agent, e.g., as a redox polymer; and (c) a mass transport limiting membrane permeable to glutamate that overcoats the glutamate-responsive active area; (ii) applying a potential to the first working electrode; (iii) obtaining a first signal at or above an oxidation-reduction potential of the glutamate-responsive active area, the first signal being proportional to a concentration of glutamate in a fluid contacting the glutamate-responsive active area; and (iv) correlating the first signal to the concentration of glutamate in the fluid. In certain embodiments, the mass transport limiting membrane can comprise a polymer, e.g., a polyurethane, or a copolymer thereof. In certain embodiments, the mass transport limiting membrane can comprise a polymer, e.g., a polyurethane, or a copolymer thereof and a second polymer, e.g., an ion-exchange polymer.

In certain embodiments, methods of the present disclosure can include: (i) exposing an analyte sensor to a fluid (e.g., bodily fluid comprising glutamate), wherein the analyte sensor comprises: (a) a sensor tail comprising at least a first working electrode; (b) a glutamate-responsive active area disposed upon a surface of the first working electrode and responsive to glutamate, where the glutamate-responsive active area comprises a glutamate oxidase and, optionally, a first polymer and/or an electron transfer agent, e.g., as a redox polymer; and (c) a mass transport limiting membrane permeable to glutamate that overcoats the glutamate-responsive active area; (ii) applying a potential to the first working electrode; (iii) obtaining a first signal at or above an oxidation-reduction potential of the glutamate-responsive active area, the first signal being proportional to a concentration of glutamate in the fluid; and (iv) correlating the first signal to the concentration of glutamate in the fluid. In certain embodiments, the mass transport limiting membrane can include a polymer, e.g., a polyurethane, or a copolymer thereof. In certain embodiments, the mass transport limiting membrane can comprise a polymer, e.g., a polyurethane, or a copolymer thereof and a second polymer, e.g., an ion-exchange polymer.

In certain embodiments, the present disclosure further provides methods for detecting glutamate and a second analyte. For example, but not by way of limitation, the method of the present disclosure can further include detecting a second analyte by providing an analyte sensor that includes a second active area and/or exposing an analyte sensor that includes a second active area to a fluid (e.g., bodily fluid comprising glutamate and the second analyte). In certain embodiments, the analyte sensor for use in a method for detecting glutamate and a second analyte can further include a second working electrode; and a second active area disposed upon a surface of the second working electrode and responsive to the second analyte differing from the first analyte, where the second active area comprises at least one enzyme responsive to the second analyte and, optionally, a second polymer and/or an electron transfer agent; wherein a portion, e.g., second portion, of the mass transport limiting membrane overcoats the second active area. Alternatively, the second active site can be covered by a second mass transport limiting membrane that is separate and/or different than the mass transport limiting membrane that overcoats the glutamate-responsive active area.

In certain embodiments, the glutamate sensors of the present disclosure can be used for up to about 10 days, about 11 days, about 12 days, about 13 days, about 14 days, about 15 days, about 16 days, about 17 days, about 18 days, about 19 days or about 20 days. In certain embodiments, the glutamate sensors of the present disclosure can be used for up to about 15 days.

The present disclosure is further illustrated by the following embodiments.

[1] An analyte sensor comprising: (i) a sensor tail comprising at least a first working electrode; (ii) a glutamate-responsive active area disposed upon a surface of the first working electrode, wherein the glutamate-responsive active area comprises a glutamate oxidase and an electron transfer agent; and (iii) a mass transport limiting membrane permeable to glutamate that overcoats at least a portion of the glutamate-responsive active area.

[2] The analyte sensor of [1], wherein the glutamate-responsive active area further comprises a polymer, wherein the glutamate oxidase and/or the electron transfer agent are crosslinked with the polymer.

[3] The analyte sensor of [1] or [2], wherein the glutamate-responsive active further comprises a stabilizer.

[4] The analyte sensor of [3], wherein the stabilizer comprises an albumin.

[5] The analyte sensor of any one of [1]-[4], wherein the mass transport limiting membrane comprises a polyurethane or a copolymer thereof.

[6] The analyte sensor of [5], wherein the mass transport limiting membrane further comprises an ion-exchange polymer or a polyvinylpyridine-based polymer.

[7] The analyte sensor of any one of [1]-[6], further comprising: (iv) a second working electrode; and (v) a second active area disposed upon a surface of the second working electrode and responsive to a second analyte differing from glutamate, wherein the second active area comprises at least one enzyme responsive to the second analyte; wherein a second portion of the mass transport limiting membrane overcoats the second active area.

[8] A method for detecting glutamate comprising: (i) providing an analyte sensor comprising: (a) a sensor tail comprising at least a first working electrode, (b) a glutamate-responsive active area disposed upon a surface of the first working electrode, wherein the glutamate-responsive active area comprises a glutamate oxidase and an electron transfer agent; and (c) a mass transport limiting membrane permeable to glutamate that overcoats at least a portion of the glutamate-responsive active area; (ii) applying a potential to the first working electrode; (iii) obtaining a first signal at or above an oxidation-reduction potential of the glutamate-responsive active area, the first signal being proportional to a concentration of glutamate in a fluid contacting the glutamate-responsive active area; and (iv) correlating the first signal to the concentration of glutamate in the fluid.

[9] The method of [8], wherein the glutamate-responsive active area further comprises a polymer.

[10] The method of [9], wherein the glutamate oxidase and/or the electron transfer agent are covalently bonded to the polymer in the glutamate-responsive active area.

[11] The method of any one of [8]-[10], wherein the glutamate-responsive active further comprises a stabilizer.

[12] The method of [11], wherein the stabilizer is an albumin.

[13] The method of any one of [8]-[12], wherein the mass transport limiting membrane comprises a polyurethane or a copolymer thereof.

[14] The method of [13], wherein the mass transport limiting membrane further comprises an ion-exchange polymer or a polyvinylpyridine-based polymer.

[15] The method of any one of [8]-[14], wherein the analyte sensor further comprises: (d) a second working electrode; and (e) a second active area disposed upon a surface of the second working electrode and responsive to a second analyte differing from glutamate, wherein the second active area comprises at least one enzyme responsive to the second analyte; wherein a second portion of the mass transport limiting membrane overcoats the second active area.

[16] The method of any one of [8]-[15], wherein the fluid is interstitial fluid.

[17] The method of any one of [8]-[16], wherein the analyte sensor is implanted in a subject at risk of having or has a neurological condition.

[18] The method of [17], wherein the neurological condition is a brain injury.

[19] The method of [18], wherein the brain injury is a traumatic brain injury.

[20] The method of [19], wherein the traumatic brain injury is a stroke and/or an intracerebral hemorrhage.

[21] The method of any one of [8]-[20], wherein the analyte sensor is implanted in a subject for about 15 days.

[22] The method of any one of [8]-[21], wherein the analyte sensor retains at least about 90% sensitivity during its use.

[23] The analyte sensor of any one of [1]-[7] for use in monitoring a subject at risk of having or has a neurological condition.

### EXAMPLES

The presently disclosed subject matter will be better understood by reference to the following Examples, which are provided as exemplary of the presently disclosed subject matter, and not by way of limitation.

### Example 1: Glutamate Sensor

The present example provides a sensor for detecting glutamate. As shown in FIG. 6, the sensor includes glutamate oxidase and a redox mediator to detect L-glutamate in a sample. The chemical composition for the glutamate-responsive active area of the sensor includes a glutamate oxidase, a stabilizer and a crosslinking agent.

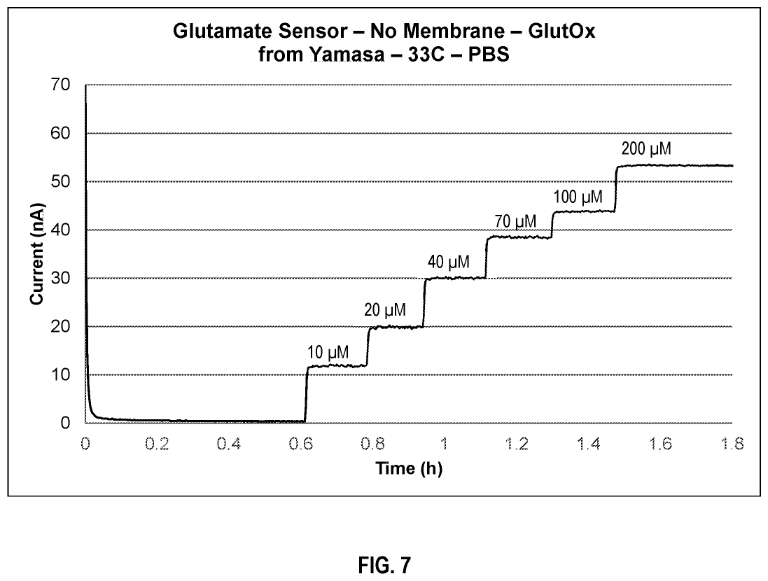

The glutamate sensors were generated by depositing a composition including the components provided in Table 1 onto a working electrode and thermally cured. The glutamate sensors were subsequently tested by consecutive additions of various glutamate concentrations in the presence of 100 mM phosphate buffered saline (PBS) at physiological pH (7.4) and at 33° C. FIG. 7, which provides the chronoamperometric response of the glutamate sensor, shows that when a concentration of glutamate was introduced to the supporting electrolyte solution covering the glutamate sensor, electrons that were generated as result of enzymatic process created a current increase for a few minutes until a stable current was achieved. The current recorded for the glutamate sensor was dependent on the glutamate concentration showing that a glutamate-responsive area that includes glutamate oxidase and a redox mediator is well suited for detecting glutamate levels in a sample.

TABLE 1

Glutamate Oxidase
HEPES
Stabilizer
Redox Mediator
Crosslinking Agent

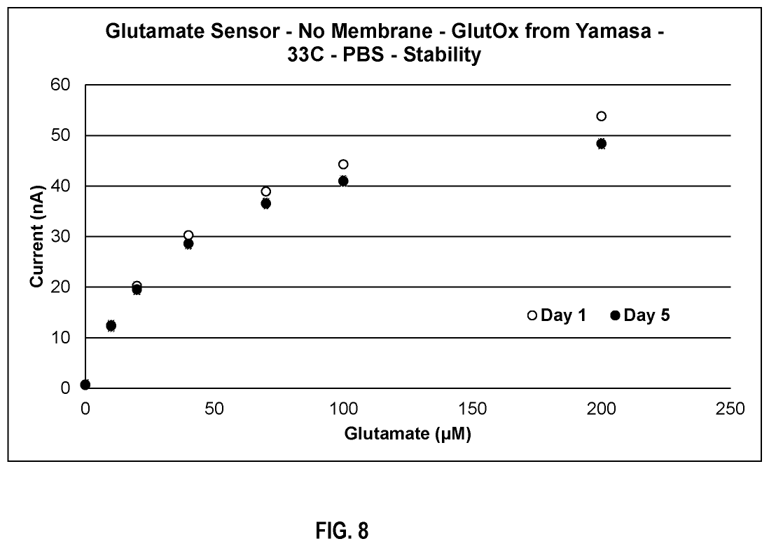

The glutamate sensors were recalibrated at different time points under the same testing conditions to evaluate sensor stability. FIG. 8 illustrates the current response versus glutamate concentration plots obtained at 2 different time points. As shown in FIG. 8, the glutamate sensors showed a 5-day stability without the presence of a mass transport limiting membrane, and less than a 10% sensitivity decrease over the time period tested was observed.

### Example 2: Mass Transport Limiting Membranes

Addition of a mass transport limiting membrane can improve a sensor's linear concentration range for the target molecule by controlling the analyte diffusion rate to the glutamate sensitive layer. This mass transport membrane can also serve as a protective agent for the glutamate-responsive area against mechanical and chemical stressors. The composition of a mass transport limiting membrane can be tailored for different analyte sensitivity targets.

Polyurethane-based membranes as mass transport limiting membranes were tested in this Example. Polyurethane-based membranes can provide some benefits to glutamate sensors over other membrane polymers. For example, polyurethane membranes do not carry positively or negatively charged functional groups like polyvinylpyridine and 10Q5. This charge neutrality allows glutamate (a molecule with one positively and 2 negatively charged groups) to diffuse easily to the glutamate-sensitivity layer to generate concentration dependent current signal. In addition, polyurethane membranes do not require crosslinker agents to form hydrogels and the density of the hydrogel is mainly defined by the concentration of the PU solution and its solvent system. With these properties, polyurethane membranes can provide wider working ranges for glutamate detection compared to polyvinylpyridine-based membranes.

A membrane comprising a polyurethane (PU) polymer (e.g., a HydroMed™ D1. or D7 polymer) or composite membranes comprising PU and an ion-exchange polymer were tested by coating the glutamate sensing layer described in Example 1 with the polymers by dip coating. Short and long side chain ion-exchange polymers such as AQUIVION® (Solvay) and NAFION® (Sigma Aldrich), respectively, were used in the composite membranes. The polymer concentration was 7.5% (w/v%) for HydroMed™ D1, 20% (w/v%) for NAFION® and 25% (w/v%) for AQUIVION®. The single layered HydroMed™ D1. membranes had a dried thickness of about 11 μm. HydroMed™ D1/AQUIVION® composite membranes, which had a three layered configuration, were generated by dip coating, and the first HydroMed™ D1. layer had a dried thickness of about 11 μm, the AQUIVION® layer had a dried thickness of about 22.5 μm and the second HydroMed™ D1. layer had a dried thickness of about 11 μm. HydroMed™ D1/NAFION® composite membranes, which had a three layered configuration, were generated by dip coating, and the first HydroMed™ D1. layer had a dried thickness of about 11 μm, the NAFION® layer had a dried thickness of about 18 μm and the second HydroMed™ D1 layer had a dried thickness of about 11 μm.

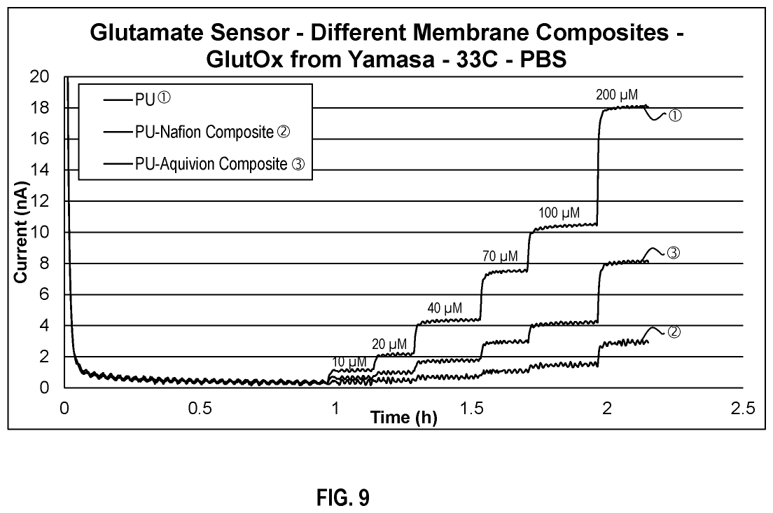

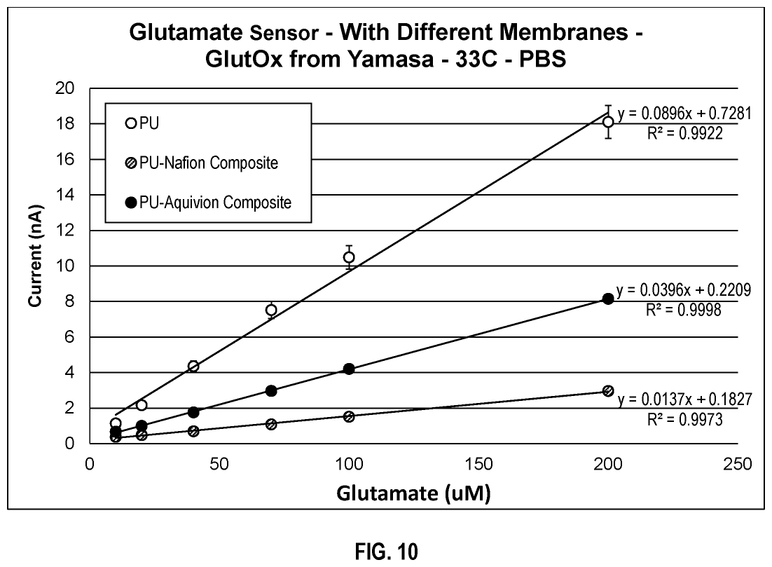

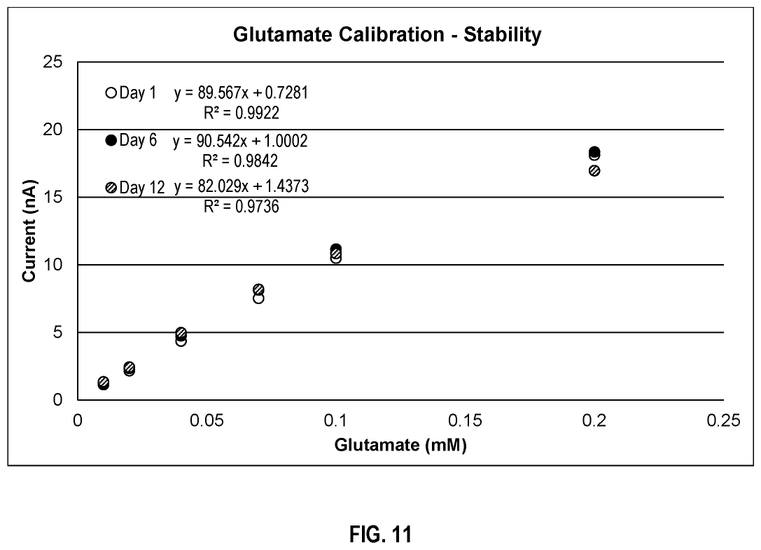

FIGS. 9 and 10 display current response curves of different membrane composites that were coated over the glutamate sensing layer. The coated glutamate sensors were tested with the same glutamate concentration range in 100 mM PBS at 33° C. As shown in FIGS. 9 and 10, the highest analyte sensitivity was obtained with a PU membrane (HydroMed™, AdvanSource Biomaterials Corp). In addition, the sensitivity of the sensor was tuned by making composite membranes comprising polyurethane and AQUIVION® or NAFION® (FIGS. 9 and 10). As shown in FIG. 11, the sensors coated with a HydroMed™ D1. membrane having a dried thickness of about 11 μm showed better stability compared to the sensors without membranes. FIG. 11 illustrates the calibration plots obtained at Day 1, 6 and 12 within the same glutamate concentration range. The sensors largely retained their sensitivity over 12 days and had a <10% loss in sensitivity.

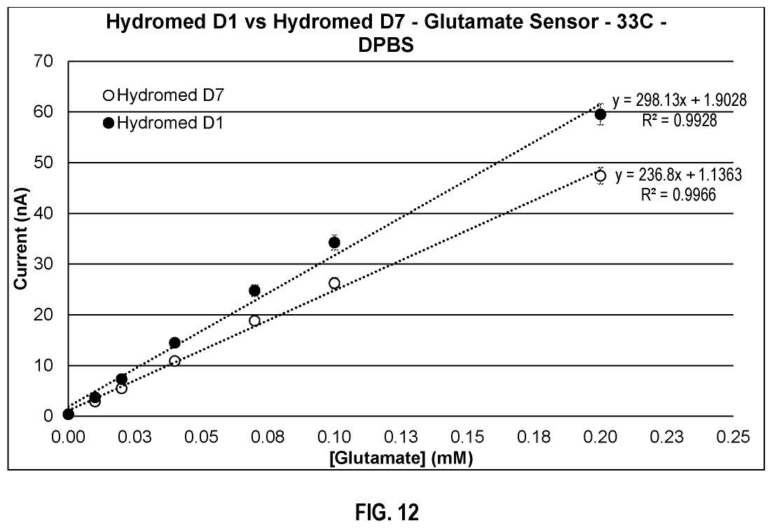

A comparison of different PU polymers was also performed. As shown in FIG. 12, the PU polymer HydroMed™ D7 was compared with the PU polymer HydroMed™ D1. As shown in FIG. 12, sensors coated with HydroMed™ D7 showed a decrease in glutamate sensitivity compared to sensors coated with HydroMed™ D1. HydroMed™ D7 shows less water absorption capacity compared to HydroMed™ D1. (30% versus 70% water absorption capacity, respectively), and without being limited to a particular theory, this change in the absorption capacity may be the reason for the decrease in the glutamate sensitivity compared to sensors coated with HydroMed™ D1. However, 10-day stability data suggested that sensors with HydroMed™ D7 membranes were more stable (<6% decrease) than the ones coated with HydroMed™ D1. (25% decrease).

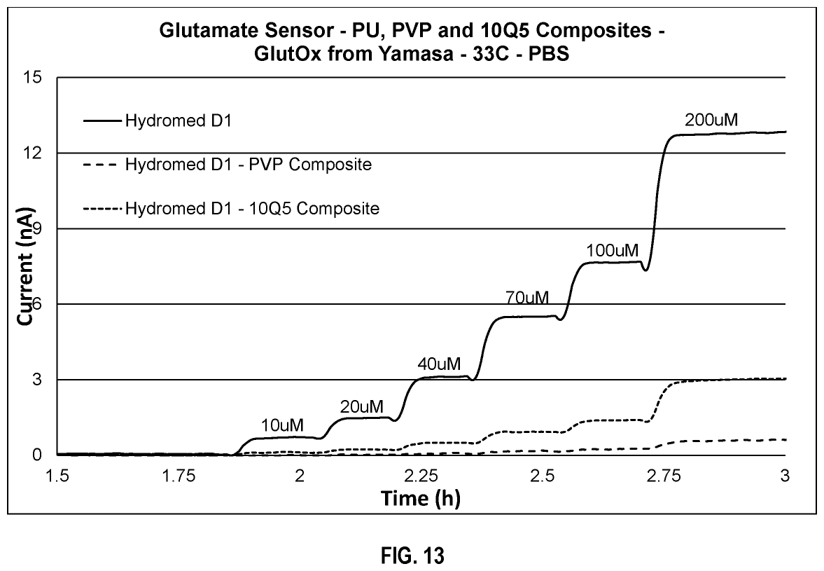

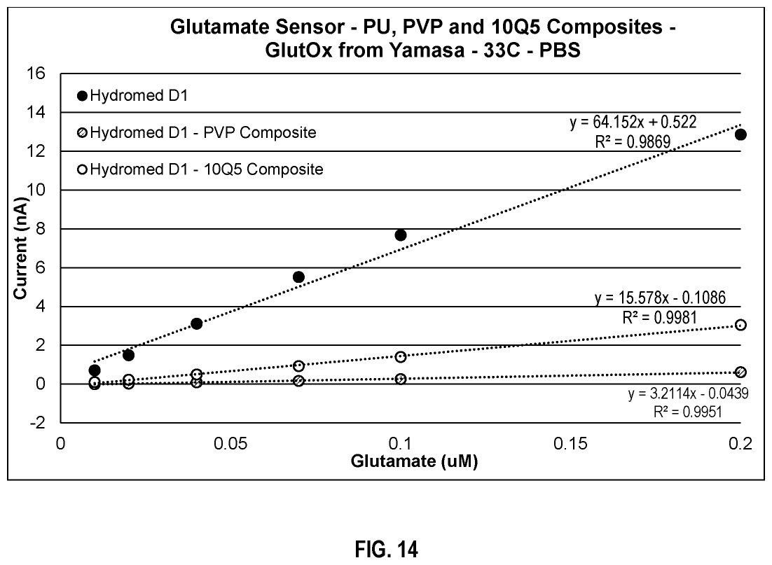

Additional PU composite membranes were tested. As discussed above, 10Q5 and PVP polymers cannot be used as single component mass transport limiting membranes because the resulting sensor will not be sensitive enough to detect physiologically relevant glutamate concentrations. However, it was tested whether composite membranes comprising PU and a polyvinylpyridine-based polymer, e.g., polyvinylpyridine (PVP) or 10Q5, can be used for coating glutamate sensors. Such glutamate sensors were generated by depositing the glutamate sensing chemistry of Example 1 on a substrate. The sensors were then coated with a 75 mg/ml HydroMed™ D1. solution followed by 65 mg/ml PVP or 80 mg/ml 10Q5 solution. Both of the PVP and 10Q5 membrane solutions contained 10% of the PEGDGE crosslinker at 5% v/v. As shown in FIGS. 13 and 14, both 10Q5 and PVP can be used to obtain functional sensors in the presence of a HydroMed™ D1. inner membrane. Although PU-10Q5 and PU-PVP composite membranes reduced glutamate sensitivity (FIGS. 13 and 14), they improved sensor stability over a 14-day continuous run. The changes in the 14-day sensitivity of the sensors were <20%, <4% and <2% for HydroMed™ D1, HydroMed™ D1-PVP and HydroMed™ D1-10Q5 membrane compositions, respectively.

The data in this Example show that PU-based membranes can improve the sensitivity and stability of glutamate sensors.

Although the presently disclosed subject matter and its advantages have been described in detail, it should be understood that various changes, substitutions and alterations can be made herein without departing from the spirit and scope of the disclosed subject matter. Moreover, the scope of the present application is not intended to be limited to the particular embodiments of the process, machine, manufacture, and composition of matter, methods and processes described in the specification.

As one of ordinary skill in the art will readily appreciate from the disclosed subject matter of the presently disclosed subject matter, processes, machines, manufacture, compositions of matter, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, methods, or steps.

Various patents, patent applications, publications, product descriptions, protocols, and sequence accession numbers are cited throughout this application, the inventions of which are incorporated herein by reference in their entireties for all purposes.

What is claimed is:

1. An analyte sensor comprising:
(i) a sensor tail comprising at least a first working electrode;
(ii) a glutamate-responsive active area disposed upon a surface of the first working electrode, wherein the glutamate-responsive active area comprises a glutamate oxidase and an electron transfer agent; and
(iii) a mass transport limiting membrane permeable to glutamate that overcoats at least a portion of the glutamate-responsive active area.

2. The analyte sensor of claim 1, wherein the glutamate-responsive active area further comprises a polymer, wherein the glutamate oxidase and/or the electron transfer agent are crosslinked with the polymer.

3. The analyte sensor of claim 1, wherein the glutamate-responsive active further comprises a stabilizer.

4. The analyte sensor of claim 3, wherein the stabilizer comprises an albumin.

5. The analyte sensor of claim 1, wherein the mass transport limiting membrane comprises a polyurethane or a copolymer thereof.

6. The analyte sensor of claim 5, wherein the mass transport limiting membrane further comprises an ion-exchange polymer or a polyvinylpyridine-based polymer. 7 The analyte sensor of claim 1, further comprising:
(iv) a second working electrode; and
(v) a second active area disposed upon a surface of the second working electrode and responsive to a second analyte differing from glutamate, wherein the second active area comprises at least one enzyme responsive to the second analyte;
wherein a second portion of the mass transport limiting membrane overcoats the second active area.

8. A method for detecting glutamate comprising:
(i) providing an analyte sensor comprising:
(a) a sensor tail comprising at least a first working electrode,
(b) a glutamate-responsive active area disposed upon a surface of the first working electrode, wherein the glutamate-responsive active area comprises a glutamate oxidase and an electron transfer agent; and
(c) a mass transport limiting membrane permeable to glutamate that overcoats at least a portion of the glutamate-responsive active area;
(ii) applying a potential to the first working electrode;
(iii) obtaining a first signal at or above an oxidation-reduction potential of the glutamate-responsive active area, the first signal being proportional to a concentration of glutamate in a fluid contacting the glutamate-responsive active area; and
(iv) correlating the first signal to the concentration of glutamate in the fluid.

9. The method of claim 8, wherein the glutamate-responsive active area further comprises a polymer.

10. The method of claim 9, wherein the glutamate oxidase and/or the electron transfer agent are covalently bonded to the polymer in the glutamate-responsive active area.

11. The method of claim 8, wherein the glutamate-responsive active further comprises a stabilizer.

12. The method of claim 11, wherein the stabilizer is an albumin.

13. The method of claim 8, wherein the mass transport limiting membrane comprises a polyurethane or a copolymer thereof.

14. The method of claim 13, wherein the mass transport limiting membrane further comprises an ion-exchange polymer or a polyvinylpyridine-based polymer.

15. The method of claim 8, wherein the analyte sensor further comprises:
(d) a second working electrode; and
(e) a second active area disposed upon a surface of the second working electrode and responsive to a second analyte differing from glutamate, wherein the second active area comprises at least one enzyme responsive to the second analyte;
wherein a second portion of the mass transport limiting membrane overcoats the second active area.

16. The method of claim 8, wherein the fluid is interstitial fluid.

17. The method of claim 8, wherein the analyte sensor is implanted in a subject at risk of having or has a neurological condition.

18. The method of claim 17, wherein the neurological condition is a brain injury.

19. The method of claim 18, wherein the brain injury is a traumatic brain injury.

20. The method of claim 19, wherein the traumatic brain injury is a stroke and/or an intracerebral hemorrhage.

21. The method of claim 8, wherein the analyte sensor is implanted in a subject for about 15 days.

22. The method of claim 8, wherein the analyte sensor retains at least about 90% sensitivity during its use.
